(19) United States
(12) Patent Application Publication
(10) Pub. No.: US 2026/0007706 A1
(43) Pub. Date: Jan. 8, 2026

(54) METHODS AND PHARMACEUTICAL COMPOSITIONS FOR THE TREATMENT AND THE PREVENTION OF ALZHEIMERS DISEASE

(71) Applicant: CORNELL UNIVERSITY, Ithaca, NY (US)

(72) Inventors: Ronald G. CRYSTAL, New York, NY (US); Stephen M. KAMINSKY, Bronx, NY (US); Dolan SONDHI, New York, NY (US)

(21) Appl. No.: 18/881,485

(22) PCT Filed: Jul. 7, 2023
(86) PCT No.: PCT/US2023/069801

§ 371 Date: Jan. 6, 2025

Related U.S. Application Data
(60) Provisional application No. 63/367,995, filed on Jul. 8, 2022.
(60) Provisional application No. 63/385,889, filed on Dec. 2, 2022.
(60) Provisional application No. 63/496,792, filed on Apr. 18, 2023.

(51) Int. Cl. A61K 35/76; A61K 31/573; A61K 38/17; A61K 48/00; A61P 25/28; C07K 14/005; C07K 14/775; C12N 15/86

(52) U.S. Cl. CPC A61K 35/76; A61K 31/573; A61K 38/17; A61K 48/005; A61P 25/28; C07K 14/005; C07K 14/775; C12N 15/86

(57) ABSTRACT

The present disclosure provides methods and compositions for the treatment of Alzheimer's disease. The methods and compositions of the present disclosure comprise AAV vectors and AAV viral vectors comprising transgene nucleic acid molecules comprising nucleic acid sequences encoding for an APOE2 polypeptide.

### CROSS-REFERENCE TO RELATED APPLICATIONS

This application claims priority to, and the benefit of, U.S. Provisional Application No. 63/367,995 filed Jul. 8, 2022, U.S. Provisional Application No. 63/385,889 filed Dec. 2, 2022, and U.S. Provisional Application No. 63/496,792 filed Apr. 18, 2023. The contents of each of which is hereby incorporated by reference in their entireties.

### SUBMISSION OF SEQUENCE LISTING IN XML FORMAT

contents of the electronic sequence listing (LEXE_010_001WO_SeqList_ST26.xml; Size: 28,886 bytes; and Date of Creation: Jul. 7, 2023) are herein incorporated by reference in its entirety.

### FIELD OF THE INVENTION

The invention relates to a method for preventing or treating Alzheimer's disease in a subject in need thereof, comprising administering to said subject a therapeutically effective amount of an AAV vector which comprises an apolipoprotein 2 (APOE2) encoding nucleic acid. Also provided are methods of producing AAV viral vectors encoding APOE2.

### BACKGROUND OF THE INVENTION

There has been a long-felt but unmet need for therapeutics effective for the treatment or prevention of Alzheimer's disease (AD). Disclosed is an AAV vector encoding the human APOE2 gene. The AAV vector is formulated for administration to individuals having Alzheimer's disease or at risk of developing Alzheimer's disease. Also provided are methods of producing APOE2 AAV vectors. The common APOE alleles (APOE4, APOE3, APOE2) are, by far, the major genetic risk-modifying factors for AD, with the APOE4 allele increasing risk and reducing the age of onset and the APOE2 allele decreasing risk and markedly delaying the age of onset. Extensive human genetic data demonstrate that APOE4 and APOE2 are co-dominant. APOE2/APOE4 heterozygotes have the normal risk of APOE3/APOE3 homozygotes, instead of having the 4-fold higher risk for AD of APOE3/APOE4 heterozygotes. APOE4 homozygotes have a markedly increased risk of developing AD (14.5-fold compared to APOE3 homozygotes), as well as an earlier age of onset for developing the disease (approximately 5 years for each APOE4 allele compared to APOE3 homozygotes). Between 45% and 50% of AD patients carry at least one APOE4 allele compared to only 15% of age-matched healthy controls. In contrast, APOE2 is a protective allele, reducing AD risk by approximately 50% (1.8-fold decreased risk) and markedly delaying the age of onset, even in the presence of the APOE4 allele. Thus, roughly equivalent expression of APOE2 cancels out the deleterious effect of the E4 allele in humans.

Amyloid burden is associated with the neuropathology of AD. Both experimental animal and clinical studies have shown that APOE genotype also predicts the timing and amount of brain Aβ peptide deposition as well as amyloid burden (APOE4>APOE3>APOE2). Intrahippocampal administration of APOE4 and APOE2 using gene transfer vectors increases and decreases, respectively, brain Aβ/amyloid burden in models of AD-related amyloidosis. Amyloid burden is one mechanism by which APOE isoforms exert their effect.

Other mechanisms include the tau pathway.

Therefore, patients homozygous for APOE4 (APOE4 homozygotes) have the highest risk for developing AD and APOE3/APOE4 heterozygotes also having an enhanced risk of developing AD. Methods and compositions of the disclosure can be used to increase APOE2 expression in the CNS of APOE4 homozygous or APOE3/APOE4 heterozygous subjects to convert their brain into a brain resembling an APOE2/APOE4 brain (i.e. low risk of developing AD), thereby reducing the risk of developing AD, treating symptoms of AD, and/or reversing symptoms of AD.

### SUMMARY OF THE INVENTION

The disclosure provides a pharmaceutical composition comprising APOE2 rAAV viral vectors; wherein the rAAV viral vector comprises an AAVrh10 capsid protein and an APOE2 rAAV vector; wherein the pharmaceutical composition comprises at least about 1.0×10¹¹ genome copies (gc)/mL to about 1.0×10¹⁴ gc/mL and wherein the pharmaceutical composition comprises less than about 40% empty rAAV capsids.

In some aspects, the pharmaceutical composition comprises less than about 40%, about 35%, about 30%, about 25%, about 20%, about 15%, about 10%, or about 5% empty rAAV capsids.

In some aspects, the pharmaceutical composition comprises at least about 1.5×10¹³ gc/mL.

In some aspects, the APOE2 rAAV viral vectors are formulated in about 1 mM potassium phosphate monobasic, 3 mM sodium phosphate dibasic, and about 155 mM sodium chloride (NaCl) at a pH of about 7.4.

In some aspects, the rAAV vector comprises in the 5′ to 3′ direction: a first AAV ITR sequence; an enhancer sequence; a promoter sequence; a chimeric intron; the nucleic acid sequence encoding an apolipoprotein 2 (APOE2) polypeptide; a polyA sequence; and a second ITR sequence.

In some aspects, the nucleic acid sequence encoding an APOE2 polypeptide comprises SEQ ID NO: 5.

In some aspects, the first ITR sequence comprises the nucleic acid sequence set forth in SEQ ID NO: 1.

In some aspects, the second ITR sequence comprises the nucleic acid sequence set forth in SEQ ID NO: 7, SEQ ID NO: 10, or SEQ ID NO: 12.

In some aspects, the enhancer sequence comprises the nucleic acid sequence set forth in SEQ ID NO: 2.

In some aspects, the promoter sequence comprises the nucleic acid sequence set forth in SEQ ID NO: 3.

In some aspects, the polyA sequence comprises the nucleic acid sequence set forth in SEQ ID NO: 6.

In some aspects, the rAAV vector comprises the nucleic acid sequence set forth in SEQ ID NO: 8 or SEQ ID NO: 11.

In some aspects, the rAAV vector is packaged as an rAAV viral vector comprising an AAV capsid protein.

In some aspects, the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh10 capsid protein. In some aspects, the AAV capsid protein is an AAVrh10 capsid protein.

The disclosure provides a pharmaceutical composition comprising APOE2 rAAV viral vectors; wherein the rAAV viral vector comprises an AAVrh10 capsid protein and an APOE2 rAAV vector; wherein the APOE2 rAAV vector comprises the nucleic acid sequence set forth in SEQ ID NO: 8 or SEQ ID NO: 11.

The disclosure provides a method of treating Alzheimer's disease in a subject in need thereof comprising administering a therapeutically effective amount of a pharmaceutical composition according to embodiments of the disclosure.

In some aspects, following administration the subject experiences an at least about 5% increase in APOE2 expression relative to a pre-administration baseline.

In some aspects, the patient is an APOE4 homozygote.

In some aspects, the pharmaceutical composition is administered via C1-C2 administration or intracisterna magna (ICM) administration.

In some aspects, the pharmaceutical composition is administered at a dose of about 5.0×10⁹ gc/mL CSF to about 5.0×10¹² gc/mL CSF. In some aspects, the pharmaceutical composition is administered at a dose of about: i) 1.4×10¹⁰ gc/mL CSF, ii) 4.4×10¹⁰ gc/mL CSF, iii) 5.0×10¹⁰ gc/mL CSF, iv) 1.4×10¹¹ gc/mL CSF, v) 1.6×10¹¹ gc/mL CSF, or vi) 5.0×10¹¹ gc/mL CSF.

In some aspects, the pharmaceutical composition is administered in a total volume of about 5 mL, about 10 mL, about 15 mL, or about 20 mL.

In some aspects, the subject experiences an at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% increase in APOE2 expression.

In some aspects, the APOE2 expression occurs in the central nervous system. In some aspects, the APOE2 expression is measured in the cerebral spinal fluid (CSF).

In some aspects, following administration of the pharmaceutical composition the expression levels of at least one of T-tau, and P-tau are reduced in the subject relative to a pre-administration baseline. In some aspects, the expression levels of T-tau, and/or P-tau are reduced by at least about 5%, at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some aspects, following administration of the pharmaceutical composition the amyloid beta 42/amyloid beta 40 (ABβ_{2/40}) ratio is increased. In some aspects, the Aβ_{42/40} ratio is increased by at least about 5%, at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some aspects, prior to treatment with the pharmaceutical composition the subject is administered an immunosuppressant. In some aspects, the immunosuppressant is prednisone. In some aspects, the prednisone is administered at a dosage of: 40 mg, once daily 1 week prior to AAV viral vector administration; 40 mg once daily for week 1 through week 2 post-AAV viral vector administration; 30 mg once daily for week 3 post-AAV viral vector administration; 20 mg once daily for week 4 post-AAV viral vector administration; 10 mg once daily for week 5 post-AAV viral vector administration; 5 mg once daily for week 6 post-AAV viral vector administration; 2.5 mg once daily for week 7 post-AAV viral vector administration; and 2.5 mg every other day for week 8 post-AAV viral vector administration.

The disclosure provides a method of producing a cellular lysate comprising rAAV viral vectors, the method comprising: (i) transfecting a cell culture comprising HEK293T cells in a transfection medium with a first plasmid encoding an APOE2 AAV vector and a second plasmid encoding AAV Rep proteins and AAV Cap proteins, wherein the ratio of second plasmid to first plasmid is 2:1; (ii) culturing the transfected HEK293T cells in the culture medium under conditions in which the transfected HEK293T cells produce recombinant adeno-associated virus (rAAV) viral vectors encoding APOE2; (iii) harvesting the transfected HEK293T cells; and (iv) lysing the transfected HEK293T cells to produce a cellular lysate comprising the rAAV viral vectors.

In some aspects, the culture medium comprises Dulbecco's Modified Eagle Medium (DMEM) with 10% fetal bovine serum (FBS). In some aspects, the transfection medium comprises serum-free DMEM and polyethylenimine (PEI).

In some aspects, the HEK293T cells are obtained after an expansion culture over about two to about five days.

In some aspects, the transfection of the first plasmid and the second plasmid occur simultaneously.

In some aspects, the HEK293T cells are present in the culture vessel at a density of between about 2.0×10³ to about 2.0×10⁶ cells/cm². In some aspects, the transfected cells are cultured for about 3 days.

In some aspects, the HEK293T cells are lysed via at least about 4 sequential freeze-thaw cycles to produce the cellular lysate.

In some aspects, the cellular lysate is further treated with a recombinant nuclease to digest any nonencapsidated DNA.

In some aspects, following DNA digestion the cellular lysate is clarified via ultracentrigufation.

In some aspects, the cellular lysate comprises from about 1.0×10⁹ to about 5.0×10¹⁴ genome copies (gc) per milliliter.

The disclosure provides a method for producing an APOE2 rAAV pharmaceutical composition, the method comprising: (i) obtaining a cellular lysate comprising rAAV viral vectors encoding APOE2; (ii) contacting a density gradient with the cellular lysate comprising rAAV viral vectors encoding APOE2 and subjecting the density gradient to centrifugation; (iii) contacting an anion exchange column with the cellular lysate comprising rAAV viral vectors encoding APOE2; (iv) eluting the rAAV viral vectors from the column; and (v) concentrating the eluted rAAV viral vectors via ultrafiltration into a formulation buffer thereby producing an APOE2 rAAV pharmaceutical composition.

In some aspects, the density gradient is an iodixanol density gradient.

In some aspects, the iodixanol gradient comprises a step-wise density gradient comprising: (i) an about 10% to about 20% iodixanol solution; (ii) an about 20% to about 30% iodixanol solution; (iii) an about 40% to about 50% iodixanol solution; and (iv) an about 50% to about 60% iodixanol solution.

In some aspects, the anion exchange column is a Q sepharose high performance strong quaternary ammonium anion exchange resin column.

In some aspects, the formulation buffer comprises phosphate buffered saline (PBS).

In some aspects, the pharmaceutical composition, following ultracentrifugation, comprises about 1.0×10¹⁰ to about 5.0×10¹³ viral genomes per milliliter.

The disclosure provides an APOE2 rAAV pharmaceutical composition produced by the method of any embodiment of the disclosure.

The disclosure provides a cellular lysate comprising rAAV viral vectors produced by the method of any embodiment of the disclosure.

The disclosure provides an rAAV vector comprising the nucleic acid sequence set forth in SEQ ID NO: 8 or SEQ ID NO: 11.

The disclosure provides an rAAV vector comprising in the 5′ to 3′ direction: a first AAV ITR sequence; an enhancer sequence; a promoter sequence; a chimeric intron; the nucleic acid sequence encoding an apolipoprotein 2 (APOE2) polypeptide; a polyA sequence; and a second ITR sequence.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

FIG. 1 is a graph depicting APOE2 levels relative to APOE4 levels in patients treated with APOE2 rAAV vectors at indicated time points.

FIG. 2 is a graph depicting APOE2 levels in in patients treated with APOE2 rAAV vectors at indicated time points.

FIG. 3 is a series of graphs depicting changes in Aβ42 (FIG. 3A), T-Tau (FIG. 3B), and P-Tau (FIG. 3C) expression in patients treated with APOE2 rAAV vectors at 12 months relative to a pre-treatment baseline.

FIG. 4A is a graph depicting the amount of viral genome copies detected in APP.PSEN1/TRE4 mice hippocampus following administration of AAVrh10 viral capsids encoding null (AAV-Rh10-NULL), APOE2 (AAV-Rh10-E2), or APOE2+hemagglutinin (HA) tag (AAV-Rh10-E2HA). The y-axis depicts viral genome copies per microgram DNA. Data represented as geometric mean ±SD. Circles stand for non-HA tagged (AAV-Rh10-E2); triangles stand for HA tagged (AAV-Rh10-E2HA) treatments.

FIG. 4B is a graph depicting APOE mRNA levels detected in APP.PSEN1/TRE4 mice hippocampus following administration of AAVrh10 viral capsids encoding null (AAV-Rh10-NULL), APOE2 (AAV-Rh10-E2), or APOE2 HA tag (AAV-Rh10-E2HA). The y-axis depicts transgene mRNA copies per microgram RNA. Data represented as geometric mean ±SD. Circles stand for non-HA tagged (AAV-Rh10-E2); triangles stand for HA (AAV-Rh10-E2HA) tagged treatments.

FIG. 4C is a graph depicting APOE protein levels detected in APP.PSEN1/TRE4 mice hippocampus following administration of AAVrh10 viral capsids encoding null (AAV-Rh10-NULL), APOE2 (AAV-Rh10-E2) or APOE2 HA tag (AAV-Rh10-E2HA). The y-axis depicts APOE protein levels expressed as ng of APOE per microgram of total protein. Data represented as geometric mean ±SEM. Circles stand for non-HA tagged (AAV-Rh10-E2), triangles stand for HA tagged (AAV-Rh10-E2HA) treatments.

FIG. 5A is a series of graphs depicting soluble and insoluble Aβ₄₂ protein levels in APP.PSEN1/TRE4 mice hippocampus following administration of AAVrh10 viral capsids encoding null (AAV-Rh10-NULL) or APOE2 (AAV-Rh10-E2). The y-axis depicts APOE levels expressed as ng of Aβ₄₂ per milligram of total protein. Data represented as geometric mean ±SEM.

FIG. 5B is a series of graphs depicting soluble and insoluble Aβ₄₂ protein levels in APP.PSEN1/TRE4 mice hippocampus following administration of AAVrh10 viral capsids encoding null (AAV-Rh10-NULL) or APOE2 (AAV-Rh10-E2). The y-axis depicts APOE levels expressed as ng of Aβ₄₂ per milligram of total protein. Data represented as geometric mean ±SEM.

FIG. 6 is a graph depicting number of beta amyloid positive staining in APP.PSEN1/TRE4 mice following administration of AAVrh10 viral capsids encoding null (AAV-Rh10-NULL), APOE2 (AAV-Rh10-E2), or APOE2 HA tag (AAV-Rh10-E2HA). Data represented as mean ±SEM. Circles stand for non-HA tagged (AAV-Rh10-E2); triangles stand for HA tagged (AAV-Rh10-E2HA) treatments.

FIG. 7 is a graph depicting fluoro jade positivity in APP.PSEN1/TRE4 mice hippocampus following administration of AAVrh10 viral capsids encoding null (AAV-Rh10-NULL), APOE2 (AAV-Rh10-E2), or APOE2 HA tag (AAV-Rh10-E2HA). Data represented as mean ±SEM. Circles stand for non-HA-tagged (AAV-Rh10-E2) and triangles stand for HA-tagged (AAV-Rh10-E2HA) treatments.

FIG. 8 is a graph depicting GFAP positivity in APP.PSEN1/TRE4 mice following administration of AAVrh10 viral capsids encoding null (AAV-Rh10-NULL), APOE2 (AAV-Rh10-E2), or APOE2 HA tag (AAV-Rh10-E2HA). Data represented as mean ±SEM. Circles stand for non-HA-tagged (AAV-Rh10-E2) and triangles stand for HA-tagged (AAV-Rh10-E2HA) treatments.

FIG. 9 is a graph depicting Ibal positivity in APP.PSEN1/TRE4 mice following administration of AAVrh10 viral capsids encoding: null (AAV-Rh10-NULL), APOE2 (AAV-Rh10-E2) or APOE2 HA tag (AAV-Rh10-E2HA). Data represented as mean ±SEM. Circles stand for non-HA-tagged (AAV-Rh10-E2) and triangles stand for HA-tagged (AAV-Rh10-E2HA) treatments.

FIG. 10 is a graph depicting X-34 stain intensity in APP.PSEN1/TRE4 mice following administration of AAVrh10 viral capsids encoding: null (AAV-Rh10-NULL), APOE2 (AAV-Rh10-E2) or APOE2 HA tag (AAV-Rh10-E2HA). Data represented as mean ±SEM. Circles stand for non-HA-tagged (AAV-Rh10-E2) and triangles stand for HA-tagged (AAV-Rh10-E2HA) treatments.

FIG. 11 is a graph depicting nesting behaviour score in APP.PSEN1/TRE4 mice following administration of AAVrh10 viral capsids encoding null (AAV-Rh10-NULL), APOE2 (AAV-Rh10-E2) or APOE2 HA tag (AAV-Rh10-E2HA. Data represented as mean ±SEM. Circles stand for non-HA-tagged (AAV-Rh10-E2) and triangles stand for HA-tagged (AAV-Rh10-E2HA) treatments.

FIG. 12 is a graph depicting Y Maze performance, a measure of spatial working memory, in APP.PSEN1/TRE4 mice following administration of AAVrh10 viral capsids encoding null (AAV-Rh10-NULL) or APOE2 (AAV-Rh10-E2). The Y-axis depicts number of entries. Data represented as mean ±SEM. Circles stand for non-HA-tagged (AAV-Rh10-E2) treatment.

FIG. 13 is a graph depicting Y Maze performance, a measure of spatial working memory, in APP.PSEN1/TRE4 mice following administration of AAVrh10 viral capsids encoding: null (AAV-Rh10-NULL) or APOE2 (AAV-Rh10-E2). The Y-axis depicts number of alterations. Data represented as mean ±SEM. Circles stand for non-HA-tagged (AAV-Rh10-E2) treatment.

FIG. 14 is a graph depicting Y Maze performance, a measure of spatial working memory, in APP.PSEN1/TRE4 mice following administration of AAVrh10 viral capsids encoding: null (AAV-Rh10-NULL) or APOE2 (AAV-Rh10-E2). The Y-axis depicts alteration percentage. Data represented as mean ±SEM. Circles stand for non-HA-tagged (AAV-Rh10-E2) treatment.

FIG. 15 is a graph depicting novel object recognition, a measure of working memory, in APP.PSEN1/TRE4 mice following administration of AAVrh10 viral capsids encoding: null (AAV-Rh10-NULL), APOE2 (AAV-Rh10-E2), or APOE2 HA tag (AAV-Rh10-E2HA. The Y-axis depicts discrimination index. Data represented as mean ±SEM. Circles stand for non-HA-tagged (AAV-Rh10-E2) and triangles stand for HA-tagged (AAV-Rh10-E2HA) treatments.

FIG. 16 is a graph depicting Barnes Maze Test performance, a hippocampal-dependent spatial memory measure, in APP.PSEN1/TRE4 mice following administration of AAVrh10 viral capsids encoding: null (AAV-Rh10-NULL) or APOE2 (AAV-Rh10-E2). The Y-axis depicts escape latency in seconds. Data represented as mean ±SEM. Circles stand for non-HA-tagged (AAV-Rh10-E2) treatment.

### DETAILED DESCRIPTION OF THE INVENTION

The disclosure provides a method of treating or preventing Alzheimer's Disease in a subject in need thereof. In some aspects, the subject having Alzheimer's disease is homozygous for APOE4 expression. In some aspects, the method comprises administering to the subject a therapeutically effective amount of a recombinant adeno associated virus (rAAV) vector which comprises a nucleic acid sequence encoding an apolipoprotein 2 (APOE2) polypeptide or a fragment thereof. In some aspects, the vector is administered in a therapeutically effective amount at a dose ranging from 5.0×10⁹ genome copies (gc)/mL CSF to about 5.0×10¹² gc/mL CSF. In some aspects, the vector is administered via C1-C2 administration or intracisterna magna (ICM) administration. In some aspects, the rAAV vector comprises the nucleic acid sequence set forth in SEQ ID NO: 8 or SEQ ID NO: 11.

### AAV Vectors

In some aspects, an isolated nucleic acid sequence comprising the nucleic acid sequence encoding apolipoprotein 2 (APOE2) can be a recombinant AAV vector (rAAV vector).

An “rAAV vector” as used herein is in reference to a vector comprising, consisting essentially of, or consisting of one or more transgene sequences and one or more AAV inverted terminal repeat sequences (ITRs). In some aspects, rAAV vectors contain one or more of an enhancer, a promoter, at least one nucleic acid that may encode at least one protein, an intronic sequence, and a poly A sequence.

In some aspects, the invention relates to an rAAV vector which comprises a apolipoprotein 2 (APOE2) encoding nucleic acid for use in the treatment or prevention of Alzheimer's disease in a subject in need thereof.

In some aspects, the invention relates to an rAAV vector which comprises a apolipoprotein 2 (APOE2) encoding nucleic acid for use in the treatment of a cardiomyopathy associated with Alzheimer's disease in a subject in need thereof.

In some aspects, the invention relates to an rAAV vector which comprises a apolipoprotein 2 (APOE2) encoding nucleic acid for reversing or stabilizing symptoms of Alzheimer's disease in a subject in need thereof.

In some aspects, the invention relates to an rAAV vector which comprises a apolipoprotein 2 (APOE2) encoding nucleic acid for improving the symptoms of Alzheimer's disease in a subject in need thereof.

In some aspects, rAAV vectors used in methods of the disclosure comprise in the 5′ to 3′ direction a first AAV ITR sequence; an enhancer sequence; a promoter sequence; a chimeric intron sequence, a nucleic acid sequence encoding APOE2; a polyA sequence; and a second ITR sequence.

In some aspects, an APOE2-encoding AAV vector of the disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 8. In some aspects, an APOE2-encoding AAV vector of the disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 11.

In some aspects, an enhancer sequence can comprise, consist essentially of, or consist of a human cytomegalovirus (CMV) enhancer sequence. A CMV enhancer sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2.

In some aspects, a promoter sequence can comprise, consist essentially of, or consist of a chicken β-actin promoter sequence. A chicken B-actin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 3.

In some aspects, a chimeric intron sequence can comprise, consist essentially of a nucleic acid sequence at least 65%, 70%, 75%, 80%, 85%, 90%, 95%, 96%, 97%, 98%, 99% or 100% (or any percentage in between) identical to SEQ ID NO: 4.

In some aspects, a polyA sequence can comprise, consist essentially of, or consist of a β-globin polyA sequence. A β-globin polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 6.

Nucleic acid sequences encoding APOE2

Apolipoprotein (APOE) is a protein involved in the metabolism of fats in the body. It is a family of proteins that binds fats and interacts with the low density lipoprotein receptor (LDLR) which is important for normal processing of triglyceride rich lipoproteins. In peripheral tissues, APOE is produced by the liver and macrophages, and mediates cholesterol metabolism. In the central nervous system, APOE is produced by astrocytes and transports cholesterol to neurons via APO receptors, which are members of the LDLR family.

APOE is synthesized as a 317 amino acid protein that is intracellularly processed to cleave an N-terminal 18 amino acid signal peptide (SEQ ID NO: 13) resulting in a mature protein 299 amino acids in length. There are three major variants of APOE: APOE2, APOE3, and APOE4. The three variants differ from one another at two positions: residue 112 (residue 130 inclusive of signal peptide) and residue 158 (residue 176 inclusive of signal peptide. The amino acid differences of the three APOE variants are set forth in Table 1. Table 1 also displays the prevalence in the global population and the relative risk for developing AD.

APOE2 is characterized in that it has a cysteine at position 112 (residue 130 including the signal peptide) and a cysteine at position 158 (residue 176 including the signal peptide). APOE3 is characterized in that it has a cysteine at position 112 (residue 130 including the signal peptide) and an arginine at position 158 (residue 176 including the signal peptide). APOE4 is characterized in that it has an arginine at position 112 (residue 130 including the signal peptide) and an arginine at position 158 (residue 176 including the signal peptide).

TABLE 1
Summary of APOE variants: APOE2, APOE3, and APOE4.

| | Amino Acid position mature | | | |
|---|---|---|---|---|
| | (Amino Acid position + | | | |
| | signal peptide) | | | Risk for |
| APOE | 112 | 158 | | developing |
| Variant | (130) | (176) | Prevalence | AD |
| APOE2 | Cys | Cys | 7% | Low |
| APOE3 | Cys | Arg | 79% | Average |
| APOE4 | Arg | Arg | Very low | High |

In some aspects, a nucleic acid sequence encoding an apolipoprotein 2 (APOEb2) polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 5.

In some aspects, the APOE2 polypeptide can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 9.

In some aspects, a mature APOE2 polypeptide lacking the signal peptide can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 14.

In some aspects, an APOE3 polypeptide comprising the signal peptide can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 17.

In some aspects, a mature APOE3 polypeptide lacking the signal peptide can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 15.

In some aspects, an APOE4 polypeptide comprising the signal peptide can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 18.

In some aspects, a mature APOE4 polypeptide lacking the signal peptide can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 16.

In some aspects, the invention provides a nucleic acid sequence comprising SEQ ID NO: 8 or SEQ ID NO: 11 or a variant thereof for treating Alzheimer's disease.

### Inverted Terminal Repeat Sequences

In some aspects, a first ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1, or complement thereof. In some aspects, a first ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 7, or complement thereof. In some aspects, a first ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 10, or complement thereof. In some aspects, a first ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 12, or complement thereof.

In some aspects, a second ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO:7, or complement thereof. In some aspects, a second ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1, or complement thereof. In some aspects, a second ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 10, or complement thereof. In some aspects, a second ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 12, or complement thereof.

### AAV Vectors

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B.

The AAV particle comprises, consists essentially of, or consists of three major viral proteins: VP1, VP2 and VP3. In some aspects, the AAV refers to the serotype AAV1, AAV2, AAV4, AAV5, AAV6, AAV7, AAV8, AAV9, AAV10, AAV11, AAV12, AAV13, AAVPHP.B, AAVrh74 or AAVrh.10.

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10, AAV11, AAV12, AAV13, AAVPHP.B, AAVrh74 and AAVrh.10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019; 4(22):e131610) and AAV-NP59 (described in detail in Paulk et al. Molecular Therapy, 2018; 26(1):289-303).

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13(1): 67-76 (2006); and the AAV-11 genome is provided in Virology, 330(2): 375-383 (2004). The sequence of the AAV rh. 74 genome is provided in U.S. Pat. No. 9,434,928. U.S. Patent No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra SP et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus poly A site is located at map position 95 of the AAV genome. The life cycle and genetics of AAV are reviewed in Muzyczka, Current Topics in Microbiology and Immunology, 158:97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958 June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

### AAV Viral Vectors

AAV vectors of the disclosure can be packaged as an AAV viral vector.

An “rAAV viral vector” refers to a viral particle composed of at least one rAAV capsid protein and an encapsidated polynucleotide AAV vector. Thus, production of an rAAV viral vector necessarily includes production of an rAAV vector. The term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3.

rAAV viral vectors useful in the practice of the present invention can be constructed utilizing methodologies well known in the art of molecular biology. Typically, AAV viral vectors carrying transgenes are assembled from polynucleotides encoding the transgene, suitable regulatory elements and elements necessary for production of viral proteins which mediate cell transduction.

The terms “Gene transfer” or “gene delivery” refer to methods or systems for reliably inserting foreign DNA into host cells. Such methods can result in transient expression of non integrated transferred DNA, extrachromosomal replication and expression of transferred replicons (e.g. episomes), or integration of transferred genetic material into the genomic DNA of host cells.

Examples of viral vectors include but are not limited to adenoviral, retroviral, lentiviral, herpesvirus and adeno-associated virus (AAV) vectors.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In one embodiment, adeno-associated viral (AAV) vectors are employed.

In other embodiments, the rAAV vector is AAV1, AAV2, AAV3, AAV4, AAV5, AAV6, AAV7, AAV8, AAV9, AAV10, AAV11, AAV12, AAV13, AAVPHP.B, AAVrh74 and AAVrh10 or any other serotypes of AAV that can infect humans, monkeys or other species.

In an exemplary embodiment, the rAAV vector is AAVrh10.

By an “rAAV vector” is meant a vector derived from an adeno-associated virus serotype, including without limitation, AAV1, AAV2, AAV3, AAV4, AAV5, AAV6, AAV7, AAV8, AAV9, AAV10, AAV11, AAV12, AAV13, AAVPHP.B, AAVrh74 and AAVrh10. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e. g by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the APOE2 gene) and a transcriptional termination region.

The control elements are selected to be functional in a mammalian cell. The resulting construct which contains the operatively linked components is bounded (5′ and 3′) with functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. In some aspects, ITR sequences of the disclosure may comprise a deletion of one or more nucleotides at one or more positions of the ITR sequence. In some aspects, deleted nucleotides in an ITR sequence can be repaired in vivo or during vector replication. The nucleotide sequences of AAV ITR regions are known. See, e.g., Kotin, 1994; Berns, KI “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV-6, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV-6, etc. Furthermore, 5′ and 3′ ITRs which flank a selected nucleotide sequence in an AAV expression vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell.

Particularly preferred are vectors derived from AAV serotypes having tropism for and high transduction efficiencies in cells of the mammalian myocardium, particularly cardiomyocytes and cardiomyocyte progenitors. A review and comparison of transduction efficiencies of different serotypes is provided in Cearley CN et al., 2008. In other non-limiting examples, preferred vectors include vectors derived from any serotypes like AAV1, AAV2, AAV3, AAV4, AA5, AAV6, AAV7, AAV8, AAV9, or AAVrh10, which have also been shown to transduce cells of cardiomyocytes.

The selected nucleotide sequence is operably linked to control elements that direct the transcription or expression thereof in the subject in vivo. Such control elements can comprise control sequences normally associated with the selected gene.

Alternatively, heterologous control sequences can be employed. Useful heterologous control sequences generally include those derived from sequences encoding mammalian or viral genes. Examples include, but are not limited to, the phophoglycerate kinase (PKG) promoter, CAG, MCK (muscle creatine kinase), the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), rous sarcoma virus (RSV) promoter, synthetic promoters, hybrid promoters, and the like. The promoters can be of human origin or from other species, including from mice. In addition, sequences derived from nonviral genes, such as the murine metallothionein gene, will also find use herein. Such promoter sequences are commercially available from, e. g. Stratagene (San Diego, CA).

Examples of heterologous promoters include the CMV promoter.

Examples of inducible promoters include DNA responsive elements for ecdysone, tetracycline, hypoxia andaufin.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e. g. U. S. Patents Nos. 5,173, 414 and 5,139,941; International Publications Nos. WO 92/01070 (published 23 Jan. 1992) and WO 93/03769 (published 4 Mar. 1993); Lebkowski et al., 1988; Vincent et al., 1990; Carter, 1992; Muzyczka, 1992; Kotin, 1994; Shelling and Smith, 1994; and Zhou et al., 1994. Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e.g. U.S. Pat. no. 5,139,941. In particular, several AAV vectors are described therein which are available from the American Type Culture Collection (“ATCC”) under Accession Numbers 53222,53223, 53224,53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. See, e. g., Edge, 1981; Nambair et al., 1984; Jay et al., 1984. In order to produce AAV virions, an AAV expression vector is introduced into a suitable host cell using known techniques, such as by transfection. A number of transfection techniques are generally known in the art. See, e.g., Graham et al., 1973, Sambrook et al. (1989) Molecular Cloning, a laboratory manual, Cold Spring Harbor Laboratories, New York, Davis etal. (1986) Basic Methods in Molecular Biology, Elsevier, and Chu et al., 1981. Particularly suitable transfection methods include calcium phosphate co-precipitation (Graham et al., 1973), direct microinjection into cultured cells (Capecchi, 1980), electroporation (Shigekawa et al., 1988), liposome mediated gene transfer (Mannino et al., 1988), lipid-mediated transduction (Felgner et al., 1987), and nucleic acid delivery using high-velocity microprojectiles (Klein et al., 1987).

AAV viral vectors of the disclosure comprise: i) an AAV vector described herein; and ii) an AAV capsid protein.

In some aspects, an AAV capsid protein can be any AAV capsid protein. In some aspects, the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein. In some aspects, the AAV capsid protein is an AAVrh. 10 capsid protein.

### Inverted Terminal Repeat Sequences

By “inverted terminal repeats” or “ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, 1994; Berns, K I “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV1, AA2, AAV3, AAV4, AAV5, AAV6, etc. Furthermore, 5′ and 3′ ITRs which flank a selected nucleotide sequence in an AAV expression vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell.

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence or an AAVrh10 ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh10 ITR sequence.

In some aspects, an rAAV vector of the present disclosure can comprise, consist essentially of, or consist of AAV2 ITR sequences. In some aspects, an rAAV vector of the present disclosure can comprise, consist essentially of, or consist of AAV2 ITR sequences or a modified AAV2 ITR sequence.

### Promoter and Enhancer Sequences

The term “promoter” and “promoter sequence” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind.

The selected nucleotide sequence, such as a frataxin-encoding nucleotide sequence, is operably linked to control elements that direct the transcription or expression thereof in the subject in vivo. Such control elements can comprise control sequences normally associated with the selected gene.

Alternatively, heterologous control sequences can be employed. Useful heterologous control sequences generally include those derived from sequences encoding mammalian or viral genes. Examples include, but are not limited to, the phophoglycerate kinase (PKG) promoter, CAG, MCK (muscle creatine kinase), the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), chicken β-actin (CBA) promoter, rous sarcoma virus (RSV) promoter, synthetic promoters, hybrid promoters, and the like. The promoters can be of human origin or from other species, including from mice. In addition, sequences derived from nonviral genes, such as the murine metallothionein gene, will also find use herein. Such promoter sequences are commercially available from, e. g. Stratagene (San Diego, CA).

Examples of heterologous promoters include the CMV promoter.

Examples of inducible promoters include DNA responsive elements for ecdysone, tetracycline, hypoxia andaufin.

An enhancer is a regulatory element that increases the expression of a target sequence.

A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) or synthetic techniques such that transcription of that gene is directed by the linked enhancer/promoter. Non-limiting examples of linked enhancer/promoter for use in the methods, compositions and constructs provided herein include a CMV enhancer linked to a CBA promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a gene (i.e. a transgene) that is under the control of a promoter with which it is spatially connected. A promoter can be positioned 5′ (upstream) or 3′ (downstream) of a gene under its control. A promoter can be positioned 5′ (upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

In some aspects, an enhancer sequence can comprise, consist essentially of, or consist of a human cytomegalovirus (CMV) enhancer sequence. A CMV enhancer sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2.

In some aspects, a promoter sequence can comprise, consist essentially of, or consist of a chicken β-actin promoter sequence. A chicken β-actin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 3.

### Methods of Treatment

The disclosure provides a method for treating or preventing Alzheimer's disease (AD) in a subject in need thereof, comprising administering to said subject a therapeutically effective amount of an rAAV vector which comprises a nucleic acid sequence encoding an APOE2polypeptide or a fragment thereof.

The disclosure provides a method for treating Alzheimer's disease in a subject in need thereof comprising administering a therapeutically effective amount of a pharmaceutical composition comprising an APOE2 rAAV viral vector.

The disclosure provides a method for treating Alzheimer's disease in a subject in need thereof comprising administering a therapeutically effective amount of a pharmaceutical composition comprising an APOE2 rAAV viral vector, wherein following administration the subject experiences an increase in APOE2 expression.

Subjects of the disclosure can express any combination of APOE variants including being APOE2 homozygotes, APOE3 homozygotes, APOE4 homozygotes,

APOE2/APOE4 heterozygotes, APOE2/APOE3 heterozygotes, or APOE3/APOE4 heterozygotes. In some aspects, subjects of the disclosure are APOE4 homozygotes.

Subjects of the disclosure can have a range of cognitive impairment associated with AD including no impairment, mild cognitive impairment (CI), or mild, moderate dementia, or severe dementia. In some aspects, subjects of the disclosure are at least 50 years old. In some aspects, subjects of the disclosure can be any age.

In some aspects, subjects of the disclosure have CSF biomarkers consistent with Alzheimer's disease. In some aspects, subjects of the disclosure are determined to be positive via amyloid-targeted positron emission tomography (PET).

In some aspects, the subject experiences an increase in APOE2 expression relative to a pre-administration baseline. Quantification of APOE2 expression can be performed according to any method known in the art. In some aspects the subject experiences an at least about 5%, at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% increase in APOE2 expression relative to a pre-administration baseline.

APOE2 expression can occur anywhere in the central nervous system including any region of the brain and the cerebral spinal fluid (CSF).

APOE2, APOE3, and APOE4 expression can be quantified according to any method known in the art. APOE2, APOE3, and APOE4 quantification can be performed using mass spectrometry (MS), western blot, chromatography, or chromatography-linked mass spectrometry (i.e. LC-MS).

In some aspects, APOE2 expression is reflected as a percentage of APOE4 expression and or total APOE expression. In some aspects, APOE2 expression is calculated as total APOE2 expression divided by APOE4 expression. This ratio can be multiplied by 100 to produce a percentage. In some aspects, APOE2 expression is calculated as total APOE2 expression divided by total APOE expression (total expression of APOE2, APOE3 and/or APOE4). This ratio can be multiplied by 100 to produce a percentage. Evaluation of APOE expression can be evaluated according to any suitable protein quantification method.

Hippocampal volume declines over time in individuals with AD at the prodromal stage and later. Hippocampal volume is usually correlated with cognitive and functional changes over time, making it a useful marker of disease progression. In some aspects, following treatment with APOE2 pharmaceutical compositions of the disclosure, a subject experiences an increase in hippocampal volume. In some aspects, following treatment with APOE2 pharmaceutical compositions of the disclosure, a subject's hippocampal volume stays the same. In some aspects, following treatment with APOE2 pharmaceutical compositions of the disclosure, a subject experiences a reduced rate of hippocampal volume decline. In some aspects, hippocampal volume decline is assessed by brain MRI.

### Cognitive Assessment

Cognitive improvement following administration of APOE2 pharmaceutical compositions can be assessed according to any method know in the art. Such assessments can include, but are not limited to, Clinical Dementia Rating (CDR), Alzheimer's Disease Assessment Scale—Cognitive Assessment (ADAS-Cog 13), or Mini-Mental State Examination.

The Clinical Dementia Rating (CDR) scale is a clinician-rated dementia staging system that tracks the progression of cognitive impairment in 6 categories (memory, orientation, judgment and problem solving, community affairs, home and hobbies, and personal care). Each category is scored on a 5-point scale in which None=0, Questionable=0.5, Mild=1, Moderate=2, and Severe=3. The global CDR score is established by clinical scoring rules and has values of 0 (no dementia), 0.5 (questionable dementia), 1 (mild dementia), 2 (moderate dementia), and 3 (severe dementia). The CDR-SB is obtained by adding the ratings in each of the 6 categories and ranges from 0 to 18 with higher scores indicative of greater impairment.

The CDR-SB will be administered at the Screening/Baseline visits before administration of an APOE2 pharmaceutical composition of the disclosure. These assessments will be used as a measure of clinical effect.

The Alzheimer's Disease Assessment Scale—Cognitive Subscale (13 items) (ADAS-Cog 13) is a structured scale that evaluates memory, orientation, attention, reasoning, language, and constructional praxis. Higher scores indicate greater impairment.

The MMSE is a brief 30-point questionnaire used to assess cognitive impairment with lower scores indicating greater impairment. The MMSE assesses 11 categories of cognition including orientation to time, memory, attention, concentration, naming, repetition, comprehension, and the ability to create a sentence and to copy two intersecting polygons.

### Biomarker Testing

APOE2 expression, following administration of an APOE2 pharmaceutical composition of the disclosure, can be evaluated in any region of the CNS. In some aspects, APOE2 expression is evaluated in the brain. In some aspects, APOE2 expression is evaluated in the CSF. Amyloid beta (Aβ) 42, Aβ42/40, T-tau, and P-tau, are considered the AD core biomarkers. It is thought that T-tau and P-tau increase after amyloid brain accumulation (amyloid cascade hypothesis). Both amyloid and tau accumulation are associated with inflammation in the brain at different times during disease progression, with amyloid driving inflammation during early accumulation (MCI cases) and tau after amyloid load in the brain is near AD levels (prodromal AD). Aβ42 is reduced in CSF of AD participants, T-tau and P-tau are increased. While not a core CSF biomarker, amyloid beta 40 is also assessed.

In some aspects, following administration of the pharmaceutical composition the expression levels of at least one of amyloid beta 42 (Aβ₄₂), amyloid beta 40 (Aβ₄₀), T-tau, and P-tau are reduced in the subject relative to a pre-administration baseline. In some aspects, following administration of the pharmaceutical composition the expression levels of at least one of amyloid beta 42 (Aβ₄₂), amyloid beta 40 (Aβ₄₀), T-tau, and P-tau are increased in the subject relative to a pre-administration baseline. In some aspects, following administration of the pharmaceutical composition the expression levels of at least one of amyloid beta 42 (Aβ₄₂), amyloid beta 40 (Aβ₄₀), T-tau, and P-tau remain constant in the subject relative to a pre-administration baseline.

In some aspects, the expression levels of Aβ₄₂, Aβ₄₀, T-tau, and/or P-tau are reduced by at least about 5%, at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some aspects, following administration of the pharmaceutical composition the expression levels of Aβ₄₂ is altered. In some aspects, following administration of the pharmaceutical composition, the expression levels of Aβ₄₂ is increased. In some aspects, following administration of the pharmaceutical composition, the expression levels of Aβ₄₂ is decreased. In some aspects, changes to the expression levels of Aβ₄₂ following administration of the pharmaceutical composition is subject-specific.

In some aspects, following administration of the pharmaceutical composition the amyloid beta 42/amyloid beta 40 (Aβ_{42/40}) ratio is altered In some aspects, following administration of the pharmaceutical composition, the Aβ_{42/40} ratio is increased. In some aspects, following administration of the pharmaceutical composition, the Aβ_{42/40} ratio is decreased. In some aspects, changes to Aβ_{42/40} ratio following administration of the pharmaceutical composition is subject-specific.

In some aspects, the Aβ_{42/40} ratio is increased by at least about 5%, at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In a particular embodiment, the gene encoded by a nucleic acid sequence in an AAV vector is the APOE2 gene.

As used herein in its broadest meaning, the term “preventing” or “prevention” refers to preventing the disease or condition from occurring in a subject which has not yet been diagnosed as having it or which does not have any clinical symptoms.

As used herein, the term “treating” or “treatment”, as used herein, means reversing, alleviating, or inhibiting the progress of the disorder or condition to which such term applies, or one or more symptoms of such disorder or condition. A “therapeutically effective amount” is intended for a minimal amount of active agent which is necessary to impart therapeutic benefit to a subject. For example, a “therapeutically effective amount” to a patient is such an amount which induces, ameliorates, stabilises, slows down the progression or otherwise causes an improvement in the pathological symptoms, disease progression or physiological conditions associated with or resistance to succumbing to a disorder.

As used herein, the term “subject” denotes a mammal, such as a rodent, a feline, a canine, and a primate. In some aspects, a subject according to the invention is a human. In the context of the present invention, a “subject in need thereof” denotes a subject, preferably a human, and more particularly a subject with a Alzheimer's disease.

As used herein, the term “gene” refers to a polynucleotide containing at least one open reading frame that is capable of encoding a particular polypeptide or protein after being transcribed and translated.

As used herein, the terms “coding sequence”, “a sequence which encodes a particular protein” or “encoding nucleic acid”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

In a particular embodiment, the invention relates to a method for preventing or treating Alzheimer's disease in a subject in need thereof, comprising administering to said subject a therapeutically effective amount of an AAV vector, AAV viral vector, or pharmaceutical composition which comprises an APOE2 encoding nucleic acid.

In a particular embodiment, the invention relates to a method for treating Alzheimer's disease in a subject in need thereof, comprising administering to said subject a therapeutically effective amount of an AAV vector, AAV viral vector, or pharmaceutical composition which comprises an APOE2 encoding nucleic acid.

In a particular embodiment, the invention relates to a method for reversing or stabilizing symptoms of Alzheimer's disease in a subject in need thereof, comprising administering to said subject a therapeutically effective amount of an AAV vector, AAV viral vector, or pharmaceutical composition which comprises an APOE2 encoding nucleic acid.

As used herein, the terms “asymptomatic” or “pre-symptomatic” denotes a subject with the disease (Alzheimer's disease) as defined by a genetic diagnosis but with no detectable clinical symptoms.

As used herein, the terms symptomatic denotes a subject with the disease (Alzheimer's disease) as defined by a genetic diagnosis and with the presence of cognitive impairment including MCI or mild, moderate, or severe dementia.

### Delivery of the Vectors

It is herein provided a method for treating Alzheimer's disease in a subject in need thereof, said method comprising: (a) providing an AAV vector as defined above, which comprises a nucleic acid sequence encoding an APOE2 polypeptide or a fragment thereof; and (b) delivering the AAV vector to the subject in need thereof and whereby APOE2 is expressed by the transduced cells at a therapeutically effective level.

The preferred doses and regimen may be determined by a physician, and depend on the age, sex, weight, of the subject, and the stage of the disease.

In some aspects, the AAV vector is administered to the central nervous system (CNS) of the subject. In some aspects, the administration is C1-C2 administration. C1-C2 administration refers to administration of the AAV vector between the first two vertebrae in the cervical spine. In some aspects, the C1-C2 administration is CT-guided. In some aspects, the administration is intracisterna magna (ICM). In some aspects, ICM administration is performed when C1-C2 administration is not feasible. In some aspects, factors that limit C1-C2 administration include aberrant arteries along needle trajectory and/or restricted dorsal subarachnoid space (i.e. <2 mm).

In some aspects, the AAV vector administration occurs over at least about 1 minute, at least about 2 minutes, at least about 3 minutes, at least about 4 minutes, at least about 5 minutes, at least about 10 minutes, at least about 20 minutes, or at least about 30 minutes, In some aspects, the IV infusion occurs over 60 minutes. In some aspects, C1-C2 administration occurs at a rate of about 1 mL/minute, of about 2 mL/minute, of about 3 mL/minute, of about 4 mL/minute, of about 5 mL/minute, of about 6 mL/minute, of about 7 mL/minute, of about 8 mL/minute, of about 9 mL/minute, or of about 10 mL/minute.

In some aspects, the volume of AAV vector administered is about 5 mL, about 10 mL, about 15 mL, about 20 mL, or about 25 mL.

In some aspects, the subject is administered an AAV vector of the disclosure at in a therapeutically effective dosage. In some aspects, the dosage is about 5.0×10⁹ genome copies (gc) per milliliter (mL) of cerebral spinal fluid (CSF) (gc/mL) to about 5.0×10¹² gc/mL CSF. In some aspects, the dosage is about 1.4×10¹⁰ gc/mL CSF. In some aspects, the dosage is about 4.4×10¹⁰ gc/mL CSF. In some aspects, the dosage is about 5.0×10¹⁰ gc/mL CSF. In some aspects, the dosage is about 1.4×10¹¹ gc/mL CSF. In some aspects, the dosage is about 1.6×10¹¹ gc/mL CSF. In some aspects, the dosage is about 5.0×10¹¹ gc/mL CSF.

In some aspects, the dosage is about 1.0×10¹⁰ gc/ml CSF, about 1.1×10¹⁰ gc/ml CSF, about 1.2×10¹⁰ gc/ml CSF, about 1.3×10¹⁰ gc/ml CSF, about 1.4×10¹⁰ gc/ml CSF, about 1.5×10¹⁰ gc/ml CSF, about 1.6×10¹⁰ gc/ml CSF, about 1.7×10¹⁰ gc/ml CSF, about 1.8×10¹⁰ gc/ml CSF, about 1.9×10¹⁰ gc/ml CSF, about 2.0×10¹⁰ gc/ml CSF, about 2.1×10¹⁰ gc/ml CSF, about 2.2×10¹⁰ gc/ml CSF, about 2.3×10¹⁰ gc/ml CSF, about 2.4×10¹⁰ gc/ml CSF, about 2.5×10¹⁰ gc/ml CSF, about 2.6×10¹⁰gc/ml CSF, about 2.7×10¹⁰ gc/ml CSF, about 2.8×10¹⁰ gc/ml CSF, about 2.9×10¹⁰ gc/ml CSF, about 3.0×10¹⁰ gc/ml CSF, about 3.1×10¹⁰ gc/ml CSF, about 3.2×10¹⁰ gc/ml CSF, about 3.3×10¹⁰ gc/ml CSF, about 3.4×10¹⁰ gc/ml CSF, about 3.5×10¹⁰ gc/ml CSF, about 3.6×10¹⁰ gc/ml CSF, about 3.7×10¹⁰ gc/ml CSF, about 3.8×10¹⁰ gc/ml CSF, about 3.9×10¹⁰ gc/ml CSF, about 4.0×10¹⁰ gc/ml CSF, about 4.1×10¹⁰ gc/ml CSF, about 4.2×10¹⁰ gc/ml CSF, about 4.3×10¹⁰ gc/ml CSF, about 4.4×10¹⁰ gc/ml CSF, about 4.5×10¹⁰ gc/ml CSF, about 4.6×10¹⁰ gc/ml CSF, about 4.7×10¹⁰ gc/ml CSF, about 4.8×10¹⁰ gc/ml CSF, about 4.9×10¹⁰ gc/ml CSF, about 5.0×10¹⁰ gc/ml CSF, about 5.1×10¹⁰ gc/ml CSF, about 5.2×10¹⁰ gc/ml CSF, about 5.3×10¹⁰ gc/ml

CSF, about 5.4×10¹⁰ gc/ml CSF, about 5.5×10¹⁰ gc/ml CSF, about 5.6×10¹⁰ gc/ml CSF, about 5.7×10¹⁰ gc/ml CSF, about 5.8×10¹⁰ gc/ml CSF, about 5.9×10¹⁰ gc/ml CSF, about 6.0×10¹⁰ gc/ml CSF, about 6.1×10¹⁰ gc/ml CSF, about 6.2×10¹⁰ gc/ml CSF, about 6.3×10¹⁰ gc/ml CSF, about 6.4×10¹⁰ gc/ml CSF, about 6.5×10¹⁰ gc/ml CSF, about 6.6×10¹⁰ gc/ml CSF, about 6.7×10¹⁰ gc/ml CSF, about 6.8×10¹⁰ gc/ml CSF, about 6.9×10¹⁰ gc/ml CSF, about 7.0×10¹⁰ gc/ml CSF, about 7.1×10¹⁰ gc/ml CSF, about 7.2×10¹⁰ gc/ml CSF, about 7.3×10¹⁰ gc/ml CSF, about 7.4×10¹⁰ gc/ml CSF, about 7.5×10¹⁰ gc/ml CSF, about 7.6×10¹⁰ gc/ml CSF, about 7.7×10¹⁰ gc/ml CSF, about 7.8×10¹⁰ gc/ml CSF, about 7.9×10¹⁰ gc/ml CSF, about 8.0×10¹⁰ gc/ml CSF, about 8.1×10¹⁰ gc/ml CSF, about 8.2×10¹⁰ gc/ml CSF, about 8.3×10¹⁰ gc/ml CSF, about 8.4×10¹⁰ gc/ml CSF, about 8.5×10¹⁰ gc/ml CSF, about 8.6×10¹⁰ gc/ml CSF, about 8.7×10¹⁰ gc/ml CSF, about 8.8×10¹⁰ gc/ml CSF, about 8.9×10¹⁰ gc/ml CSF, about 9.0×10¹⁰ gc/ml CSF, about 9.1×10¹⁰ gc/ml CSF, about 9.2×10¹⁰ gc/ml CSF, about 9.3×10¹⁰ gc/ml CSF, about 9.4×10¹⁰ gc/ml CSF, about 9.5×10¹⁰ gc/ml CSF, about 9.6×10¹⁰ gc/ml CSF, about 9.7×10¹⁰ gc/ml CSF, about 9.8×10¹⁰ gc/ml CSF, or about 9.9×10¹⁰ gc/ml CSF or any dosage in between.

In some aspects, about 1.0×10¹¹ gc/ml CSF, about 1.1×10¹¹ gc/ml CSF, about 1.2×10¹¹ gc/ml CSF, about 1.3×10¹¹ gc/ml CSF, about 1.4×10¹¹ gc/ml CSF, about 1.5×10¹¹ gc/ml CSF, about 1.6×10¹¹ gc/ml CSF, about 1.7×10¹¹ gc/ml CSF, about 1.8×10¹¹ gc/ml CSF, about 1.9×10¹¹ gc/ml CSF, about 2.0×10¹¹ gc/ml CSF, about 2.1×10¹¹ gc/ml CSF, about 2.2×10¹¹ gc/ml CSF, about 2.3×10¹¹ gc/ml CSF, about 2.4×10¹¹ gc/ml CSF, about 2.5×10¹¹ gc/ml CSF, about 2.6×10¹¹ gc/ml CSF, about 2.7×10¹¹ gc/ml CSF, about 2.8×10¹¹ gc/ml CSF, about 2.9×10¹¹ gc/ml CSF, about 3.0×10¹¹ gc/ml CSF, about 3.1×10¹¹ gc/ml CSF, about 3.2×10¹¹ gc/ml CSF, about 3.3×10¹¹ gc/ml CSF, about 3.4×10¹¹ gc/ml CSF, about 3.5×10¹¹ gc/ml CSF, about 3.6×10¹¹ gc/ml CSF, about 3.7×10¹¹ gc/ml CSF, about 3.8×10¹¹ gc/ml CSF, about 3.9×10¹¹ gc/ml CSF, about 4.0×10¹¹ gc/ml CSF, about 4.1×10¹¹ gc/ml CSF, about 4.2×10¹¹ gc/ml CSF, about 4.3×10¹¹ gc/ml CSF, about 4.4×10¹¹ gc/ml CSF, about 4.5×10¹¹ gc/ml CSF, about 4.6×10¹¹ gc/ml CSF, about 4.7×10¹¹ gc/ml CSF, about 4.8×10¹¹ gc/ml CSF, about 4.9×10¹¹ gc/ml CSF, about 5.0×10¹¹ gc/ml CSF, about 5.1×10¹¹ gc/ml CSF, about 5.2×10¹¹ gc/ml CSF, about 5.3×10¹¹ gc/ml CSF, about 5.4×10¹¹ gc/ml CSF, about 5.5×10¹¹ gc/ml CSF, about 5.6×10¹¹ gc/ml CSF, about 5.7×10¹¹ gc/ml CSF, about 5.8×10¹¹ gc/ml CSF, about 5.9×10¹¹ gc/ml CSF, about 6.0×10¹¹ gc/ml CSF, about 6.1×10¹¹ gc/ml CSF, about 6.2×10¹¹ gc/ml CSF, about 6.3×10¹¹ gc/ml CSF, about 6.4×10¹¹ gc/ml CSF, about 6.5×10¹¹ gc/ml CSF, about 6.6×10¹¹ gc/ml CSF, about 6.7×10¹¹ gc/ml CSF, about 6.8×10¹¹ gc/ml CSF, about 6.9×10¹¹ gc/ml CSF, about 7.0×10¹¹ gc/ml CSF, about 7.1×10¹¹ gc/ml CSF, about 7.2×10¹¹ gc/ml CSF, about 7.3×10¹¹ gc/ml CSF, about 7.4×10¹¹ gc/ml CSF, about 7.5×10¹¹ gc/ml CSF, about 7.6×10¹¹ gc/ml CSF, about 7.7×10¹¹ gc/ml CSF, about 7.8×10¹¹ gc/ml CSF, about 7.9×10¹¹ gc/ml CSF, about 8.0×10¹¹ gc/ml CSF, about 8.1×10¹¹ gc/ml CSF, about 8.2×10¹¹ gc/ml CSF, about 8.3×10¹¹ gc/ml CSF, about 8.4×10¹¹ gc/ml CSF, about 8.5×10¹¹ gc/ml CSF, about 8.6 ×10¹¹ gc/ml CSF, about 8.7×10¹¹ gc/ml CSF, about 8.8×10¹¹ gc/ml CSF, about 8.9×10¹¹ gc/ml CSF, about 9.0×10¹¹ gc/ml CSF, about 9.1×10¹¹ gc/ml CSF, about 9.2×10¹¹ gc/ml CSF, about 9.3×10¹¹ gc/ml CSF, about 9.4×10¹¹ gc/ml CSF, about 9.5×10¹¹ gc/ml CSF, about 9.6×10¹¹ gc/ml CSF, about 9.7×10¹¹ gc/ml CSF, about 9.8×10¹¹ gc/ml CSF, or about 9.9×10¹¹ gc/ml CSF or any dosage in between.

In some aspects, the dosage is about 1.0×10¹² gc/ml CSF, about 1.1×10¹² gc/ml CSF, about 1.2×10¹² gc/ml CSF, about 1.3×10¹² gc/ml CSF, about 1.4×10¹² gc/ml CSF, about 1.5×10¹² gc/ml CSF, about 1.6×10¹² gc/ml CSF, about 1.7×10¹² gc/ml CSF, about 1.8×10¹² gc/ml CSF, about 1.9×10¹² gc/ml CSF, about 2.0×10¹² gc/ml CSF, about 2.1×10¹² gc/ml CSF, about 2.2×10¹² gc/ml CSF, about 2.3×10¹² gc/ml CSF, about 2.4×10¹² gc/ml CSF, about 2.5×10¹² gc/ml CSF, about 2.6×10¹² gc/ml CSF, about 2.7×10¹² gc/ml CSF, about 2.8×10¹² gc/ml CSF, about 2.9×10¹² gc/ml CSF, about 3.0×10¹² gc/ml CSF, about 3.1×10¹² gc/ml CSF, about 3.2×10¹² gc/ml CSF, about 3.3×10¹² gc/ml CSF, about 3.4×10¹² gc/ml CSF, about 3.5×10¹² gc/ml CSF, about 3.6×10¹² gc/ml CSF, about 3.7×10¹² gc/ml CSF, about 3.8×10¹² gc/ml CSF, about 3.9×10¹² gc/ml CSF, about 4.0×10¹² gc/ml CSF, about 4.1×10¹² gc/ml CSF, about 4.2×10¹² gc/ml CSF, about 4.3×10¹² gc/ml CSF, about 4.4×10¹² gc/ml CSF, about 4.5×10¹² gc/ml CSF, about 4.6×10¹² gc/ml CSF, about 4.7×10¹² gc/ml CSF, about 4.8×10¹² gc/ml CSF, about 4.9×10¹² gc/ml CSF, about 5.0×10¹² gc/ml CSF, about 5.1×10¹² gc/ml CSF, about 5.2×10¹² gc/ml CSF, about 5.3×10¹² gc/ml CSF, about 5.4×10¹² gc/ml CSF, about 5.5×10¹² gc/ml CSF, about 5.6×10¹² gc/ml CSF, about 5.7×10¹² gc/ml CSF, about 5.8×10¹² gc/ml CSF, about 5.9×10¹² gc/ml CSF, about 6.0×10¹² gc/ml CSF, about 6.1×10¹² gc/ml CSF, about 6.2×10¹² gc/ml CSF, about 6.3×10¹² gc/ml CSF, about 6.4×10¹² gc/ml CSF, about 6.5×10¹² gc/ml CSF, about 6.6×10¹² gc/ml CSF, about 6.7×10¹² gc/ml CSF, about 6.8×10¹² gc/ml CSF, about 6.9×10¹² gc/ml CSF, about 7.0×10¹² gc/ml CSF, about 7.1×10¹² gc/ml CSF, about 7.2×10¹² gc/ml CSF, about 7.3×10¹² gc/ml CSF, about 7.4×10¹² gc/ml CSF, about 7.5×10¹² gc/ml CSF, about 7.6×10¹² gc/ml CSF, about 7.7×10¹² gc/ml CSF, about 7.8×10¹² gc/ml CSF, about 7.9×10¹² gc/ml CSF, about 8.0×10¹² gc/ml CSF, about 8.1×10¹² gc/ml CSF, about 8.2×10¹² gc/ml CSF, about 8.3×10¹² gc/ml CSF, about 8.4×10¹² gc/ml CSF, about 8.5×10¹² gc/ml CSF, about 8.6×10¹² gc/ml CSF, about 8.7×10¹² gc/ml CSF, about 8.8×10¹² gc/ml CSF, about 8.9×10¹² gc/ml CSF, about 9.0×10¹² gc/ml CSF, about 9.1×10¹² gc/ml CSF, about 9.2×10¹² gc/ml CSF, about 9.3×10¹² gc/ml CSF, about 9.4×10¹² gc/ml CSF, about 9.5×10¹² gc/ml CSF, about 9.6×10¹² gc/ml CSF, about 9.7×10¹² gc/ml CSF, about 9.8×10¹² gc/ml CSF, or about 9.9×10¹² gc/ml CSF or any dosage in between.

In some aspects, the dosage of 1.4×10¹⁰ gc/mL CSF, ii) 4.4×10¹⁰ gc/mL CSF, and 1.4×10¹¹ gc/mL CSF is quantified by ddPCR.

In some aspects, dose of: i) 5.0×10¹⁰ gc/mL CSF, ii) 1.6×10¹¹ gc/mL CSF, or iii) 5.0×10¹¹ gc/mL CSF is quantified by qPCR.

In some aspects, quantitative PCR (qPCR) doses can be converted to digital droplet PCR (ddPCR) doses. A digital droplet PCR (ddPCR) assay can be used to ensure precision and accuracy of the titration method used for administration of rAAV vectors, AAV vectors, or pharmaceutical compositions of the disclosure. Following assessment of an initial lot of APOE2 AAV viral capsid, a qPCR to ddPCR conversion factor of 3.6 was assigned enabling the conversion of qPCR doses to ddPCR doses. The skilled artisan will appreciate that that the conversion factor can be adjusted depending on variance in lot of AAV viral capsid. In some aspects, AAV vectors of the disclosure are administered at a total dosage

of about 1.0×10¹⁰ gc to about 1.0×10¹⁶ gc. In some aspects, AAV vectors of the disclosure are administered at a total dosage of about 1.0×10¹¹ gc to about 1.0×10¹⁵ gc. In some aspects, AAV vectors of the disclosure are administered at a total dosage of about 1.0×10¹² gc to about 9.9×10¹⁴ gc. In some aspects, AAV vectors of the disclosure are administered at a total dosage of about 1.0×10¹² gc, about 2.0×10¹² gc, about 3.0×10¹² gc, about 4.0×10¹² gc, about 5.0×10¹² gc, about 6.0×10¹² gc, about 7.0×10¹² gc, about 8.0×10¹² gc, about 9.0×10¹² gc, about 1.0×10¹³ gc, about 2.0×10¹³ gc, about 3.0×10¹³ gc, about 4.0×10¹³ gc, about 5.0×10¹³ gc, about 6.0×10¹³ gc, about 7.0×10¹³ gc, about 8.0×10¹³ gc, about 9.0×10¹³ gc, about 1.0×10¹⁴ , about 2.0×10¹⁴ gc, about 3.0×10¹⁴ gc, about 4.0×10¹⁴ gc, about 5.0×10¹⁴ gc, about 6.0×10¹⁴ gc, about 7.0×10¹⁴ gc, about 8.0×10¹⁴ gc, about 9.0×10¹⁴ gc, or about 1.0×10¹⁵ or any dosage in between.

In some aspects, AAV vectors of the disclosure are administered at a total dosage of about 1.0×¹⁴ gc, about 1.1×¹⁴ gc, about 1.2×¹⁴ gc, about 1.3×¹⁴ gc, about 1.4×¹⁴ gc, about 1.5×¹⁴ gc, about 1.6×¹⁴ gc, about 1.7×¹⁴ gc, about 1.8×¹⁴ gc, about 1.9×¹⁴ gc, or about 2.0×¹⁴ gc or any dosage in between. In some aspects, AAV vectors of the disclosure are administered at a total dosage of about 1.4×¹⁴ gc.

In some aspects, the therapeutically effective dosage of the AAV vector of the disclosure is administered as a fixed dosage. In some aspects a fixed dosage is expressed as a total number of genome copies (gc).

In some aspects, AAV vectors of the disclosure are administered at a fixed dosage of about 1.0×¹⁰ gc to about 1.0×10¹⁶ gc. In some aspects, AAV vectors of the disclosure are administered at a fixed dosage of about 1.0×10¹³ gc to about 1.0×10¹⁵ gc. In some aspects, AAV vectors of the disclosure are administered at a fixed dosage of about 1.0×10¹⁴ gc to about 9.0×10¹⁴ gc. In some aspects, AAV vectors of the disclosure are administered at a fixed dosage of about 1.0×10¹⁴ , about 2.0×10¹⁴ gc, about 2.1×10¹⁴ gc, about 2.2×10¹⁴ gc, about 2.3×10¹⁴ gc, about 2.4×10¹⁴ gc, about 2.5×10¹⁴ gc, about 2.6×10¹⁴ gc, about 2.7×10¹⁴ gc, about 2.8×10¹⁴ gc, about 2.9×10¹⁴ gc, about 3.0×10¹⁴ gc, about 4.0×10¹⁴ gc, about 5.0×10¹⁴ gc, about 6.0×10¹⁴ gc, about 7.0×10¹⁴ gc, about 8.0×10¹⁴ gc, or about 9.0×10¹⁴ gc, or any dosage in between.

In some aspects, AAV vectors of the disclosure are administered at a fixed dosage of about 1.0×10¹⁴ gc, about 1.1×10¹⁴ gc, about 1.2×10¹⁴ gc, about 1.3×10¹⁴ gc, about 1.4×10¹⁴ gc, about 1.5×10¹⁴ gc, about 1.6×10¹⁴ gc, about 1.7×10¹⁴ gc, about 1.8×10¹⁴ gc, about 1.9×10¹⁴ gc, or about 2.0×10¹⁴ gc or any dosage in between. In some aspects, AAV vectors of the disclosure are administered at a fixed dosage of about 2.5×10¹⁴ gc. In some aspects, AAV vectors of the disclosure are administered at a fixed dosage of about 1.4×10¹⁴ gc.

Any dosage form or method of calculating said dosage form of the AAV vectors of the disclosure is contemplated herein. In some aspects, dosages are based on the mass and/or volume of the brain. In some aspects, dosages are based on the weight of the subject. In some aspects, dosages are calculated using a qPCR titer method. In some aspects, dosages are calculated using a ddPCR titer method.

In some aspects, the therapeutically effective dosage can be tailored for each AAV capsid serotype. In some aspects, the therapeutically effective dosage is tailored to account for differences in CNS tropism for distinct AAV capsid serotypes. In some aspects, the therapeutically effective dosage is tailored to account for differences in liver tropism for distinct AAV capsid serotypes. In some aspects, the therapeutically effective dosage is tailored to account for differences in cardiac tropism for distinct AAV capsid serotypes.

In some aspects, the subject is administered a single dose of an AAV vector. In some aspects, the subject is further administered a second, third, fourth, or fifth dosage of the AAV vector. In some aspects, second and subsequent administrations of AAV vector can be at a different dosage from the first dosage.

In some aspects, the dosage is measured by quantitative polymerase chain reaction (qPCR) titer. In some aspects, the dosage is measured by droplet digital polymerase chain reaction (ddPCR) titer.

In some aspects, the subject is further administered an immunosuppressant along with AAV vector administration. The immunosuppressant can be any immunosuppressant and/or corticosteroid known in the art. Immunosuppressants can be dosed at any amount at any schedule or interval. Immunosuppressants may be administered to improve patient safety, minimize host immune response to AAV-based therapies, and/or enhance therapeutic efficacy of AAV-based therapies. In some aspects, the subject is further administered prednisone along with AAV vector administration.

In some aspects, the prednisone is administered at a dosage of:
40 mg, once daily about 1 week prior to AAV viral vector administration;
40 mg once daily for week 1 through week 2 post-AAV viral vector administration;
30 mg once daily for week 3 post-AAV viral vector administration;
20 mg once daily for week 4 post-AAV viral vector administration;
10 mg once daily for week 5 post-AAV viral vector administration;
5 mg once daily for week 6 post-AAV viral vector administration;
2.5 mg once daily for week 7 post-AAV viral vector administration; and
2.5 mg every other day for week 8 post-AAV viral vector administration.

In some aspects, if evidence of host immune response is observed following AAV vector administration, the dose of immunosuppressant can be held constant (i.e. not tapered as described above) or increased. In some aspects, a maximum dose of prednisone is about 60 mg. Once evidence of host immune response subsides or decreases, tapering of the immunosuppressant can be initiated.

In some aspects, the subject is further administered a proton-pump inhibitor during prednisone use.

In some aspects, the invention relates to a vector which comprises an APOE2 encoding nucleic acid for use in the treatment or prevention of Alzheimer's disease in a subject wherein the AAV vector is delivered to the subject in need thereof and wherein APOE2 is expressed by the transduced cells at a therapeutically effective level.

In a particular embodiment, the invention relates to a vector which comprises an APOE2 encoding nucleic acid for reversing symptoms of Alzheimer's disease in a subject in need thereof wherein the AAV vector is delivered to the subject in need thereof and wherein APOE2 is expressed by the transduced cells at a therapeutically effective level.

### Non-Viral Vectors

In a particular embodiment, the vector use according to the invention is a non-viral vector. Typically, the non-viral vector may be a plasmid which includes nucleic acid sequences encoding the APOE2 gene, or variants thereof, as described above.

### Pharmaceutical Compositions

In some aspects, the invention concerns a pharmaceutical composition for preventing or treating Alzheimer's disease in a subject in need thereof, which comprises a therapeutically effective amount of an AAV vector which comprises an APOE2 encoding nucleic acid.

The disclosure provides a pharmaceutical composition comprising APOE2 rAAV viral vectors; wherein the rAAV viral vectors comprises an AAVrh10 capsid protein and an APOE2 rAAV vector.

In some aspects, the pharmaceutical composition comprises at least about 1.0×10¹¹ gc/mL to about 1.0×10¹⁵ gc/mL of the APOE2 rAAV vector. In some aspects, the pharmaceutical composition comprises at least about 1.0×10¹¹ gc/mL to about 1.0×10¹⁴ gc/mL of the APOE2 rAAV vector. In some aspects, the pharmaceutical composition comprises at least about 1.0×10¹² gc/mL, about 1.5×10¹² gc/mL, about 2.0×10¹² gc/mL, about 2.5×10¹² gc/mL, about 3.0×10¹² gc/mL, about 3.5×10¹² gc/mL, about 4.0×10¹² gc/mL, about 4.5×10¹² gc/mL, about 5.0×10¹² gc/mL, about 5.5×10¹² gc/mL, about 6.0×10¹² gc/mL, about 6.5×10¹² gc/mL, about 7.0×10¹² gc/mL, about 7.5×10¹² gc/mL, about 8.0×10¹² gc/mL, about 8.5×10¹² gc/mL, about 9.0×10¹² gc/mL, about 9.5×10¹² gc/mL, about 1.0×10¹³ gc/mL, about 1.5×10¹³ gc/mL, about 2.0×10¹³ gc/mL, about 2.5×10¹³ gc/mL, about 3.0×10¹³ gc/mL, about 4.0×10¹³ gc/mL, about 4.5×10¹³ gc/mL, about 5.0×10¹³ gc/mL, about 5.5×10¹³ gc/mL, about 6.0×10¹³ gc/mL, about 6.5×10¹³ gc/mL, about 7.0×10¹³ gc/mL, about 7.5×10¹³ gc/mL, about 8.0×10¹³ gc/mL, about 8.5×10¹³ gc/mL, about 9.0×10¹³ gc/mL, about 9.5×10¹³ gc/mL, or any number in between of the APOE2 rAAV vector. In some aspects, the pharmaceutical composition comprises at least about 1.5×10¹³ gc/mL of the APOE2 rAAV vector.

In some aspects, the APOE2 rAAV pharmaceutical composition comprises less than about 50% empty rAAV capsids. In some aspects, the APOE2 rAAV pharmaceutical composition comprises less than about 40% empty rAAV capsids. In some aspects, the APOE2 rAAV pharmaceutical composition comprises less than about 15% empty rAAV capsids.

In some aspects, an APOE2 rAAV pharmaceutical composition disclosed herein comprises less than about 15% empty rAAV capsids. In some aspects, an APOE2 rAAV pharmaceutical composition comprises less than about 50% empty rAAV capsids, less than about 45% empty rAAV capsids, less than about 40% empty rAAV capsids, less than about 39% empty rAAV capsids, less than about 38% empty rAAV capsids, less than about 37% empty rAAV capsids, less than about 36% empty rAAV capsids, less than about 35% empty rAAV capsids, less than about 34% empty rAAV capsids, less than about 33% empty rAAV capsids, less than about 32% empty rAAV capsids, less than about 31% empty rAAV capsids, less than about 30% empty rAAV capsids, less than about 29% empty rAAV capsids, less than about 28% empty rAAV capsids, less than about 27% empty rAAV capsids, less than about 26% empty rAAV capsids, less than about 25% empty rAAV capsids, less than about 24% empty rAAV capsids, less than about 23% empty rAAV capsids, less than about 22% empty rAAV capsids, less than about 21% empty rAAV capsids, less than about 20% empty rAAV capsids, less than about 19% empty rAAV capsids, less than about 18% empty rAAV capsids, less than about 17% empty rAAV capsids, less than about 16% empty rAAV capsids, less than about 15% empty rAAV capsids, less than about 14% empty rAAV capsids, less than about 13% empty rAAV capsids, less than about 12% empty rAAV capsids, less than about 11% empty rAAV capsids, less than about 10% empty rAAV capsids, less than about 9% empty rAAV capsids, less than about 8% empty rAAV capsids, less than about 7% empty rAAV capsids, less than about 6% empty rAAV capsids, less than about 5% empty rAAV capsids, less than about 4% empty rAAV capsids, less than about 3% empty rAAV capsids, less than about 2% empty rAAV capsids, or less than about 1% empty rAAV capsids. In some aspects, an APOE2 rAAV pharmaceutical composition comprises from about 1% to about 10% empty rAAV capsids.

In some aspects, an APOE2 rAAV pharmaceutical composition comprises from about 1% to about 40% empty rAAV capsids. In some aspects, an APOE2 rAAV pharmaceutical composition comprises from about 1% to about 35% empty rAAV capsids. In some aspects, an APOE2 rAAV pharmaceutical composition comprises from about 1% to about 30% empty rAAV capsids. In some aspects, an APOE2 rAAV pharmaceutical composition comprises from about 1% to about 25% empty rAAV capsids. In some aspects, an APOE2 rAAV pharmaceutical composition comprises from about 1% to about 20% empty rAAV capsids. In some aspects, an APOE2 rAAV pharmaceutical composition comprises from about 1% to about 15% empty rAAV capsids. In some aspects, an APOE2 rAAV pharmaceutical composition comprises from about 1% to about 10% empty rAAV capsids. In some aspects, an APOE2 rAAV pharmaceutical composition comprises from about 2% to about 8% empty rAAV capsids. In some aspects, an APOE2 rAAV pharmaceutical composition comprises less than or equal to about 6% empty rAAV capsids, about 5% empty rAAV capsids, about 4% empty rAAV capsids, about 3% empty rAAV capsids, about 2% empty rAAV capsids, or about 1% empty rAAV capsids. In some aspects, the number of empty rAAV capsids is below the limit of detection. In some aspects, the percentage of empty rAAV capsids is determined as a percentage of total rAAV capsids, e.g., electron microscopy.

In some aspects, the percentage of empty rAAV capsids is determined as a percentage of total rAAV capsids, e.g., using analytical ultracentrifugation (AUC). In some aspects, the AUC is sedimentation velocity AUV (SV-AUC). In some aspects, these low percentages of empty rAAV particles improve efficacy of treatment and/or reduce adverse events (e.g., inflammatory responses, liver injury) after administration to a subject, e.g., as compared to administering compositions having higher percentage empty rAAV particles. In some aspects, the methods of preparing rAAV compositions disclosed herein provide these low percentages of empty rAAV particles, as compared to the levels of empty rAAV particles produced in other methods, e.g., those not using the production and/or the purification methods described herein.

In some aspects, an APOE2 rAAV pharmaceutical composition disclosed herein comprises at least 50% full rAAV particles. In some aspects, an APOE2 rAAV pharmaceutical composition disclosed herein comprises at least 60% full rAAV particles. In some aspects, an APOE2 rAAV pharmaceutical composition disclosed herein comprises at least 70% full rAAV particles. In some aspects, an APOE2 rAAV pharmaceutical composition disclosed herein comprises at least 80% full rAAV particles. In some aspects, an APOE2 rAAV pharmaceutical composition comprises at least 85% full rAAV particles, at least 90% full rAAV particles, at least 95% full rAAV particles, or at least about 99% full rAAV particles. In some aspects, an APOE2 rAAV pharmaceutical composition comprises 100% full rAAV particles.

APOE2 rAAV pharmaceutical compositions of the disclosure are prepared such that residual host cell DNA is removed. In some aspects, the amount of residual host cell DNA is below a predetermined value determined to be necessary for a safe and efficacious treatment suitable for administration to a subject.

APOE2 rAAV pharmaceutical compositions of the disclosure are prepared such that residual host cell protein is removed. In some aspects, the amount of residual host cell protein is below a predetermined value determined to be necessary for a safe and efficacious treatment suitable for administration to a subject.

APOE2 rAAV pharmaceutical compositions of the disclosure are prepared such that residual Adenovirus early region 1A (E1a DNA) is removed. In some aspects, the amount of E1a DNA is below a predetermined value determined to be necessary for a safe and efficacious treatment suitable for administration to a subject.

APOE2 rAAV pharmaceutical compositions of the disclosure are prepared such that residual SV40 Large T Antigen DNA (SV40 LTA) DNA is removed. In some aspects, the amount of SV40 LTA DNA is below a predetermined value determined to be necessary for a safe and efficacious treatment suitable for administration to a subject.

APOE2 rAAV pharmaceutical compositions of the disclosure are prepared such that residual non-encapsidated rAAV vector plasmid DNA is removed. In some aspects, the amount of non-encapsidated rAAV vector plasmid DNA is below a predetermined value determined to be necessary for a safe and efficacious treatment suitable for administration to a subject. In some aspects, the rAAV vector plasmid DNA is plasmid DNA that that is not encoding between the ITR regions.

APOE2 rAAV pharmaceutical compositions of the disclosure are prepared such that residual polyethylenimine (PEI) is removed. In some aspects, the amount of PEI is below a predetermined value determined to be necessary for a safe and efficacious treatment suitable for administration to a subject.

APOE2 rAAV pharmaceutical compositions of the disclosure are prepared such that residual benzonase is removed. In some aspects, the amount of benzonase is below a predetermined value determined to be necessary for a safe and efficacious treatment suitable for administration to a subject.

APOE2 rAAV pharmaceutical compositions of the disclosure are prepared such that residual bovine serum albumin (BSA) is removed. In some aspects, the amount of BSA is below a predetermined value determined to be necessary for a safe and efficacious treatment suitable for administration to a subject.

APOE2 rAAV pharmaceutical compositions of the disclosure are prepared such that residual iodixanol is removed. In some aspects, the amount of iodixanol is below a predetermined value determined to be necessary for a safe and efficacious treatment suitable for administration to a subject.

APOE2 rAAV pharmaceutical compositions of the disclosure are prepared such that replication competent AAV (rcAAV) is removed. In some aspects, the amount of rcAAV is below a predetermined value determined to be necessary for a safe and efficacious treatment suitable for administration to a subject.

By a “therapeutically effective amount” is meant a sufficient amount of the AAV vector of the invention to treat Alzheimer's disease at a reasonable benefit/risk ratio applicable to any medical treatment.

It will be understood that the single dosage or the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range per adult per day. The therapeutically effective amount of the vector according to the invention that should be administered, as well as the dosage for the treatment of a pathological condition with the number of viral or non-viral particles and/or pharmaceutical compositions of the invention, will depend on numerous factors, including the age and condition of the patient, the severity of the disturbance or disorder, the method and frequency of administration and the particular peptide to be used.

The presentation of the pharmaceutical compositions that contain the AAV vector according to the invention may be in any form that is suitable for the selected mode of administration, for example, C1-C2 administration or ICM administration.

In the pharmaceutical compositions of the present invention for administration to the CNS, the active principle, alone or in combination with another active principle, can be administered in a unit administration form, as a mixture with conventional pharmaceutical supports, to animals and human beings.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The AAV vector according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

Multiple doses can also be administered.

In some aspects, pharmaceutical compositions comprising rAAV viral vectors of the disclosure are formulated with more or more excipients suitable for administration to a subject in need thereof by any suitable method of administration. In some aspects, the one or more excipients include a phosphate buffer and a salt.

In some aspects, the phosphate buffer comprises potassium phosphate monobasic and sodium phosphate dibasic. In some aspects, the potassium phosphate monobasic is used at a concentration of about 0.01 mM to about 100 mM. In some aspects, the potassium phosphate monobasic at a concentration of about 0.1 mM, about 0.25 mM, about 0.5 mM, about 0.75 mM, about 1 mM, about 1.25 mM, about 1.5 mM, about 2 mM, about 2.5 mM, about 3 mM, about 3.5 mM, about 4 mM, about 4.5 mM, about 5 mM, about 6 mM, about 7 mM, about 8 mM, about 9 mM, about 10 mM, about 15 mM, about 20 mM, or about 30 mM, or any concentration in between. In some aspects, the potassium phosphate monobasic is used at a concentration of about 1 mM. In some aspects, the sodium phosphate dibasic is used at a concentration of about 0.01 mM to about 100 mM. In some aspects, the sodium phosphate dibasic at a concentration of about 0.5 mM, about 1 mM, about 1.5 mM, about 2 mM, about 2.5 mM, about 3 mM, about 3.5 mM, about 4 mM, about 4.5 mM, about 5 mM, about 6 mM, about 7 mM, about 8 mM, about 9 mM, about 10 mM, about 15 mM, about 20 mM, or about 30 mM, or any concentration in between. In some aspects, the sodium phosphate dibasic is used at a concentration of about 3 mM.

In some aspects, the salt is sodium chloride. In some aspects, the sodium chloride is at a concentration of about 0.01 mM to 1 M. In some aspects, the sodium chloride is at a concentration of about 25 mM to 300 mM. In some aspects, the sodium chloride is at a concentration of about 100 mM, about 110 mM, about 120 mM, about 130 mM, about 140 mM, about 150 mM, about 155 mM, about 160 mM, about 170 mM, about 180 mM, about 190 mM, or about 200 mM. In some aspects, the sodium chloride is at a concentration of about 155 mM.

In some aspects, pharmaceutical compositions are formulated at a pH suitable for administration to a subject. In some aspects, the pH of the pharmaceutical composition is at about 6.0 to about 9.0. In some aspects, the pH of the pharmaceutical composition is about 7.0, about 7.1 about 7.2, about 7.3, about 7.4, about 7.5, about 7.6, about 7.7, about 7.8, about 7.9, or about 8.0. In some aspects, the pH of the pharmaceutical composition is about 7.4.

Pharmaceutical compositions of the disclosure can be administered to a subject in any volume deemed appropriate by an attending physician and can vary depending on the specific needs of each individual subject. In some aspects, pharmaceutical compositions of the disclosure including pre-packaged pharmaceutical compositions can be diluted in an appropriate volume of fluid prior to administration. In some aspects, if the pharmaceutical composition of the disclosure is diluted, the volume of the diluted pharmaceutical composition administered to the subject is equivalent to the dosages of rAAV viral particles provided herein.

In other words, if the volume of pharmaceutical composition is increased, thereby diluting the concentration of rAAV viral particles, the patient will still be administered the same total number of either genome copies of rAAV vector or number of rAAV viral capsids as specified by the dosages disclosed herein.

In some aspects, the volume of the subject's CSF determines the final volume of the pharmaceutical composition. In some aspects, CSF volume is measured by magnetic resonance imagine (MRI).

In some aspects, the pharmaceutical composition is administered in a total volume of about 5 mL, about 10 mL, about 15 mL, about 20 mL, about 25 mL, about 30 mL, about 35 mL, about 40 mL, about 45 mL, or about 50 mL, or any volume in between.

### Manufacture of APOE2 AAV Viral Vectors

The disclosure provides methods of producing rAAV vectors and rAAV viral vectors encoding APOE2. In some aspects, rAAV viral vectors are derived from a cellular lysate produced by cell culture and purification methods described herein.

The disclosure provides a method of producing a cellular lysate comprising obtaining a culture vessel comprising rAAV viral vectors, the method comprising: (i) HEK293T cells in a culture medium; (ii) transfecting the HEK293T cells in a transfection medium with a first plasmid encoding an APOE2 AAV vector and a second plasmid encoding AAV Rep proteins and AAV Cap proteins, wherein the ratio of second plasmid to first plasmid is 2:1; (iii) culturing the transfected HEK293T cells in the culture medium under conditions which the transfected HEK293T cells produce recombinant adeno-associated virus (rAAV) viral vectors encoding APOE2; (iv) harvesting the transfected HEK293T cells; (v) lysing the transfected HEK293T cells to produce a cellular lysate comprising the rAAV viral vectors.

Culture media of the disclosure can be any culture media capable of culturing mammalian cells including but not limited to HEK293T cells. In some aspects, the culture medium comprises Dulbecco's Modified Eagle Medium (DMEM) with 10% fetal bovine serum (FBS). Cells can be expanded for any length of time necessary to produce a desirable number of cells. In some aspects, the HEK293T cells are obtained after an expansion culture over about two to about five days.

Transfection can be performed in any suitable media. In some aspects, the transfection medium comprises serum-free DMEM and polyethylenimine (PEI).

The transfection of the two plasmids can occur in any order. In some aspects, the transfection of the first plasmid and the second plasmid occur simultaneously.

In some aspects, prior to transfection, the HEK293T cells are present in the culture vessel at a density of between about 2.0×10⁴ to about 2.0×10⁶ cells/cm². In some aspects, the HEK293T cells are present at a density of about 2.0×10⁵ cells/cm².

Following transfection, cells can be cultured for any suitable length of time to produce a desired quantity of APOE2 rAAV viral vectors. In some aspects, transfected cells are cultured for about 1 day to about 7 days. In some aspects, transfected cells are cultured for about 3 days.

Following harvest of transfected cells, cells comprising APOE2 rAAV viral vectors are lysed to produce a cellular lysate comprising APOE2 rAAV viral vectors. Cell lysis can be performed by any suitable method including freeze-thaw, mechanical lysis, and lysis by chemical agents such as a detergent. In some aspects, the HEK293T cells are lysed via at least about 4 sequential freeze-thaw cycles to produce the cellular lysate.

Cellular lysates of the disclosure can be treated to remove any residual nonencapsidated viral or cellular DNA. In some aspects, the cellular lysate is further treated with a recombinant nuclease to digest any viral DNA or cellular DNA. In some aspects, the recombinant nuclease is benzonase. In some aspects, DNA digestion is performed in the presence of magnesium chloride.

Following DNA digestion, the lysate comprising APOE2 rAAV viral vectors is clarified. Clarification can be performed according to any method known in the art. In some aspects, clarification is performed by centrifugation or ultracentrifugation. Clarification of the cellular lysate removes cell debris and unbroken cells.

Following clarification, the number of rAAV vectors or rAAV viral vectors in the cellular lysate can be quantified. In some aspects, the cellular lysate comprises from about 1.0×10⁹ to about 5.0×10¹⁴ genome copies (gc) per milliliter.

The disclosure further provides a method for producing an APOE2 rAAV pharmaceutical composition, the method comprising: (i) obtaining a cellular lysate comprising rAAV viral vectors encoding APOE2; (ii) contacting a density gradient with the cellular lysate comprising rAAV viral vectors encoding APOE2 and subjecting the density gradient to centrifugation; (iii) contacting a chromatography column with the cellular lysate comprising rAAV viral vectors encoding APOE2; (iv) eluting the rAAV viral vectors from the column; (v) concentrating the eluted rAAV viral vectors via ultrafiltration into a formulation buffer thereby producing an APOE2 rAAV pharmaceutical composition.

Density gradients of the disclosure can be any suitable density gradient. Density gradients provide a means of separating components in a mixture by their size and or molecular weight. Density gradient purification can be used to separate empty AAV capsids from AAV capsids comprising APOE2 rAAV vectors. In some aspects, the density gradient is an iodixanol density gradient.

In some aspects, the iodixanol gradient comprises a step-wise density gradient comprising: (i) an about 10% to about 20% iodixanol solution; (ii) an about 20% to about 30% iodixanol solution; (iii) an about 40% to about 50% iodixanol solution; and (iv) an about 50% to about 60% iodixanol solution. In some aspects, the iodixanol gradient comprises a step-wise density gradient comprising: (i) an about 15% iodixanol solution; (ii) an about 25% iodixanol solution; (iii) an about 40% iodixanol solution; and (iv) an about 54% iodixanol solution.

In some aspects, alternative means of purification can be used in place of density gradient purification. In some aspects, chromatography can be used. In some aspects, ion exchange chromatography can be used.

Chromatography can be used to purify rAAV viral vectors following density gradient purification. In some aspects, the chromatography is anion exchange chromatography. In some aspects, the anion exchange column is a Q sepharose high performance strong quaternary ammonium anion exchange resin column.

Following chromatographic purification, APOE2 rAAV viral vectors are buffer exchanged into a formulation buffer via ultrafiltration thereby producing a pharmaceutical composition of the disclosure. In some aspects, the formulation buffer comprises phosphate buffered saline (PBS).

In some aspects, the pharmaceutical composition, following ultracentrifugation, comprises about 1.0×10¹⁰ to about 5.0×10¹³ genome copies (viral genomes) per milliliter. In some aspects, the pharmaceutical composition, following ultracentrifugation, comprises about 1.5×10¹³ genome copies (vial genomes) per milliliter.

### EXAMPLES

The invention will be further illustrated by the following figures and examples. However, these examples and figures should not be interpreted in any way as limiting the scope of the present invention.

### Example 1: Administration of APOE2 rAAV Vectors to AD Patients Results in Increased APOE2 Expression and Changes in AD Biomarker Expression

APOE4 homozygous patients having Alzheimer's Disease were administered AAV vectors encoding human APOE2 (LX1001) as part of a phase ½ trial. Administration was via injection at C1-C2 under CT guidance. C1-C2 administration is preferred, but ICM administration can be used where C1-C2 administration is not practical or possible. Patients were administered LX1001 at a dosage of 5.0×10¹⁰ gc/ml CSF (determined by qPCR) or 1.4×10¹⁰ (as determined by ddPCR). Total CSF volume of administered patients ranged from 313 to 490 mL. Following administration no serious adverse events were observed. Adverse events of transient headache occurred in ⅖ subjects and were considered related to the administration procedure. No other adverse events occurred in more than one subject.

### Patient Population

Eligibility criteria for trial participation includes: a) greater than or equal to 50 years in age, homozygous for APOE4 expression, mild cognitive impairment, mild to moderate dementia with positive amyloid PET and biomarkers consistent with Alzheimer's disease.

### Endpoints

### Primary Endpoint

Safety

### Secondary Endpoint

Conversion of APOE4 in CSF to APOE2

### Other Secondary Endpoints

CSF biomarker levels/expression: Aβ42, T Tau and P Tau
Amyloid PET scan
Quantitative MRI
Cognitive Testing

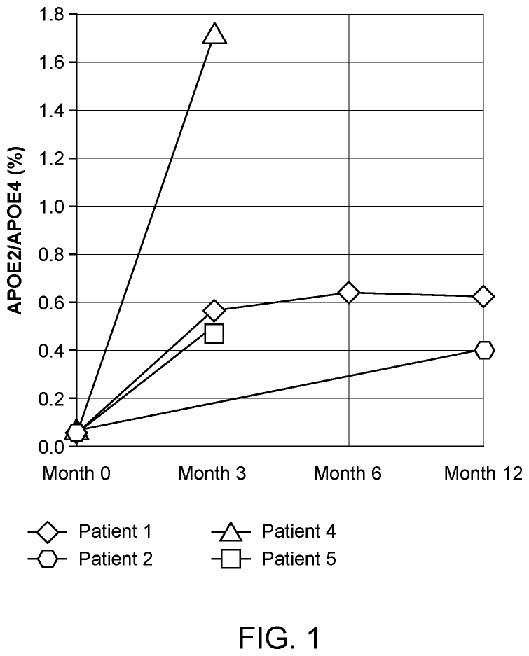

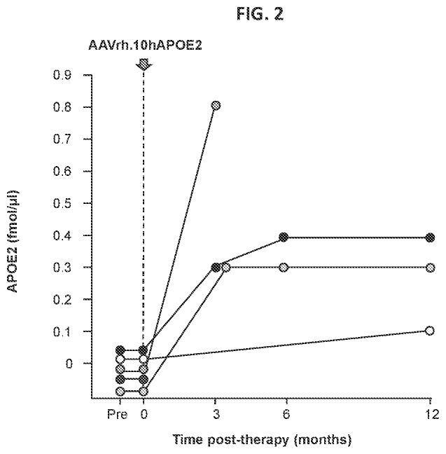

Expression of APOE2 following administration of LX1001 was evaluated at fixed time points and compared to a pre-administration baseline of APOE4 expression. APOE2/APOE4 expression was evaluated in the CSF of patients at 3, 6, and 12 months (FIG. 1 and FIG. 2). APOE2/APOE4 expression was calculated as total APOE2 expression divided by total APOE4 expression multiplied by 100 yielding a percent increase in APOE2 expression relative to APOE4 expression (FIG. 1). Patients all experienced increases in APOE2 expression following treatment with LX1001. APOE2 expression was demonstrated in all patients with follow up data, and was sustained out to 12 months (FIG. 2).

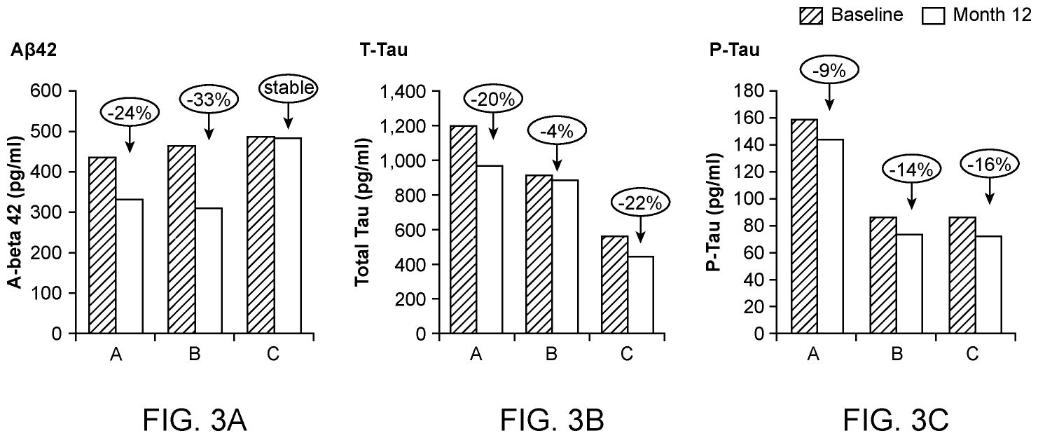

AD biomarkers were also evaluated following LX1001 administration. Aβ42, T-Tau, and P-Tau expression was measured at 2, 6, and 12 months post-LX1001 administration and compared to a pre-treatment baseline (FIG. 3). Biomarker expression was measured in the CSF. Following treatment with LX1001, treated patients generally experienced reductions in Aβ42 (FIG. 3A), T-Tau (FIG. 3B), and P-Tau (FIG. 3C) expression.

### Example 2: In Vivo Studies of AAV Vectors Encoding APOE2 Polypeptides

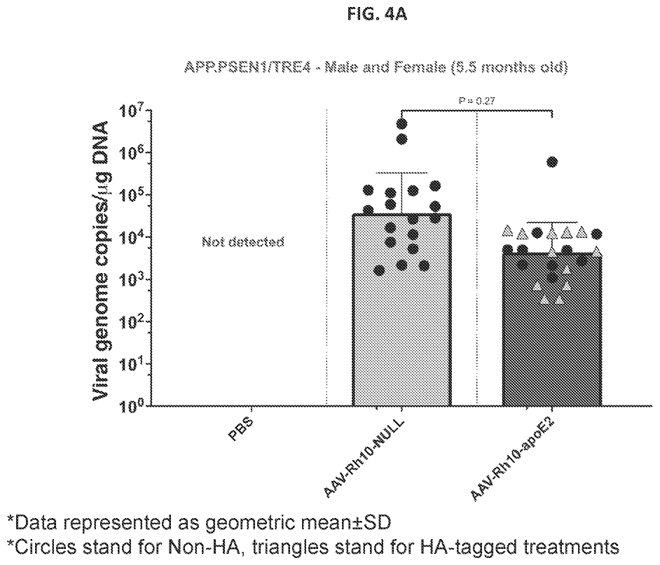

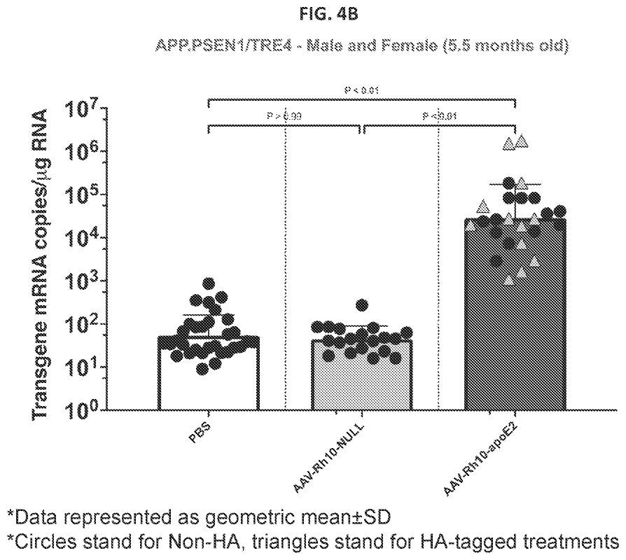

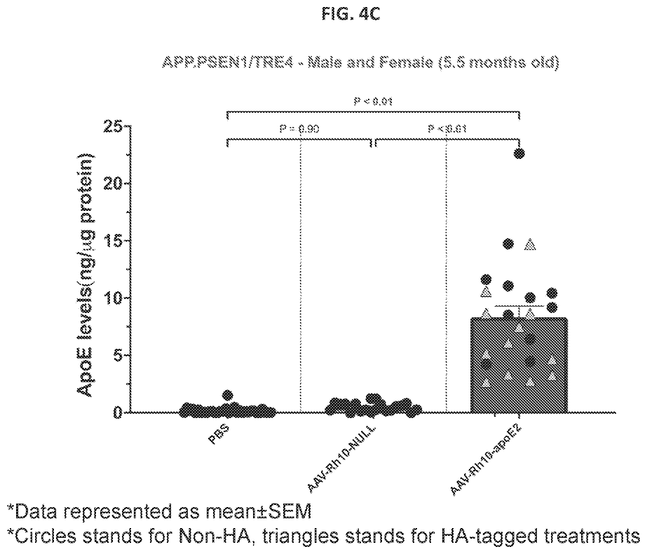

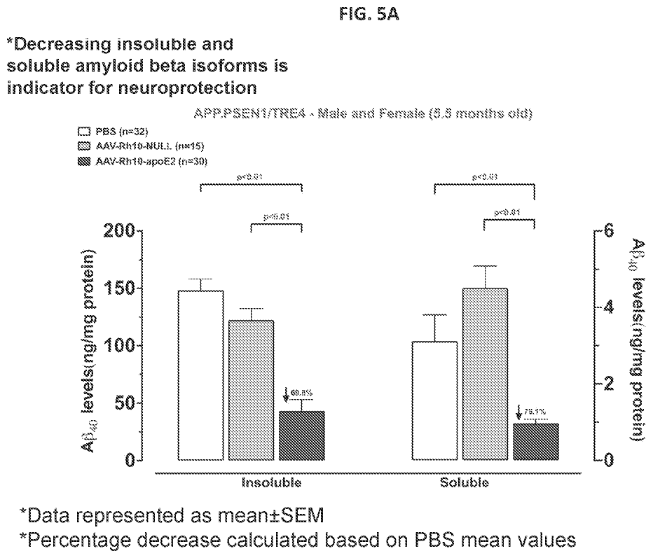

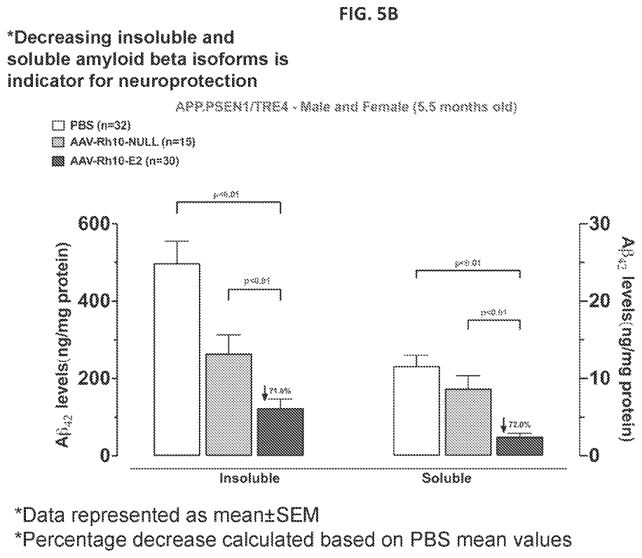

The hypothesis that AAVrh. 10-mediated expression of the combined human APOE2 will effectively protect against the development of Alzheimer's disease was evaluated. AAVrh. 10vhAPOE2C was tested in the APP.PSEN1/TRE4 amyloid plaque mouse model which has a humanized APOE4 gene, an amyloid plaque promoting presenilin mutation and a mutant amyloid precursor protein (Neurobiology of Aging 2016 44:159). AAVrh.10APOE2 and AAHrh.10 null vector was administered to the hippocampus (2×10¹⁰ genome copies) of APP.PSEN/APOE4 mice at age 2.5 months with assessment at 5.5 months post-administration. APP.PSEN/APOE4 mice administered PBS were used as controls. The AAVrh. 10hAPOE2 treated cohort had a marked increase in hippocampal human APOE2 protein compared to PBS (FIG. 4C). AAVrh.10hAPOE2 administration reduced the levels of both soluble and insoluble amyloid peptide β42 and soluble and insoluble amyloid peptide β40, respectively compared to the PBS cohort (FIG. 5A and FIG. 5B). The amount of viral genome copies in mouse brain following administration was evaluated (FIG. 4A). The amount of APOE mRNA was also determined (FIG. 4B).

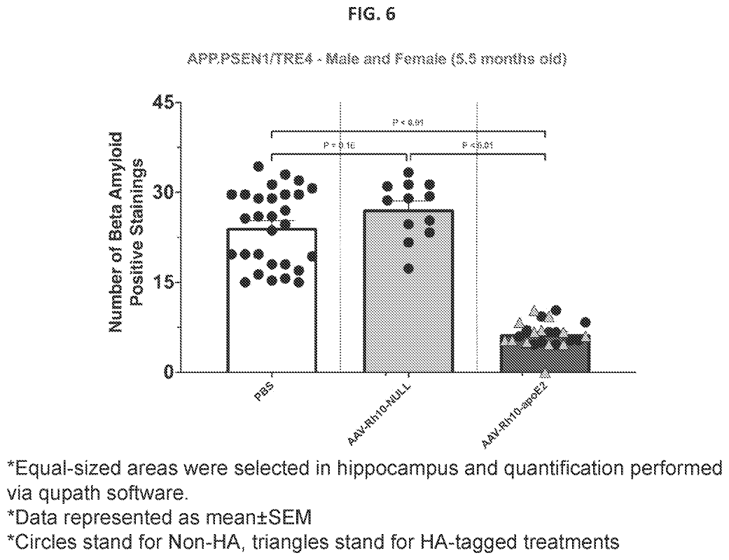

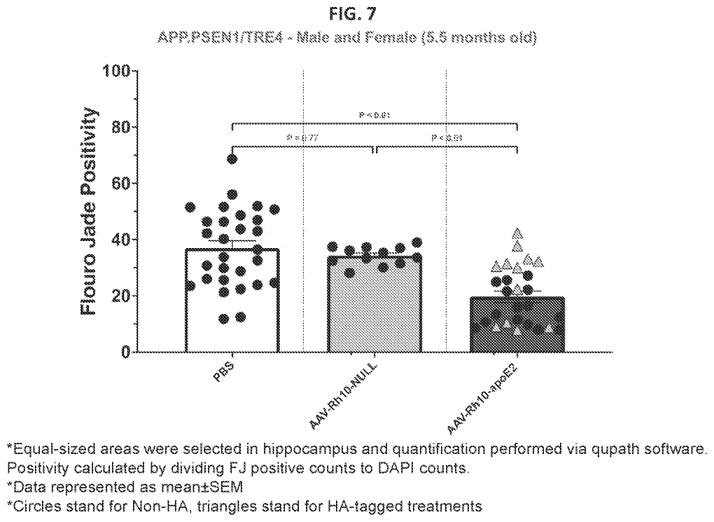

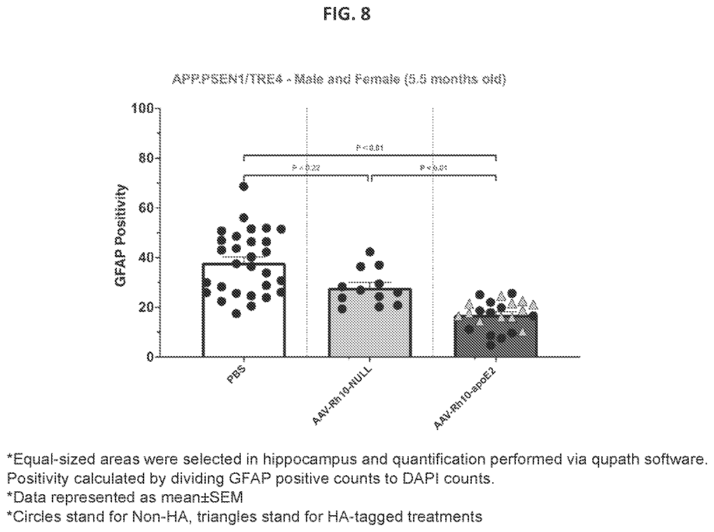

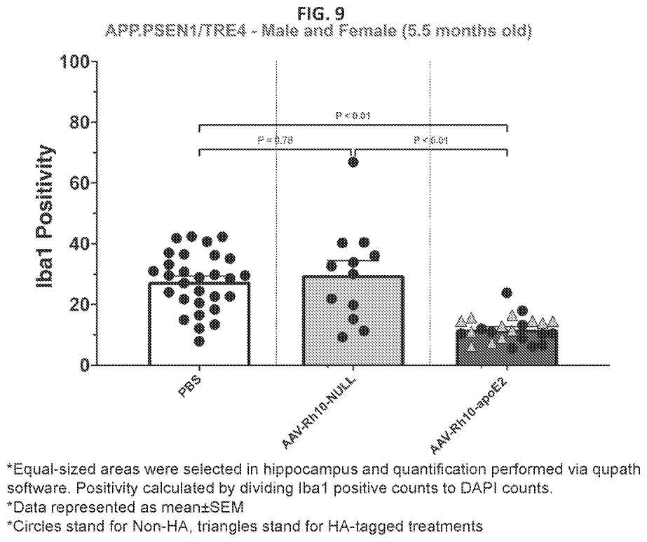

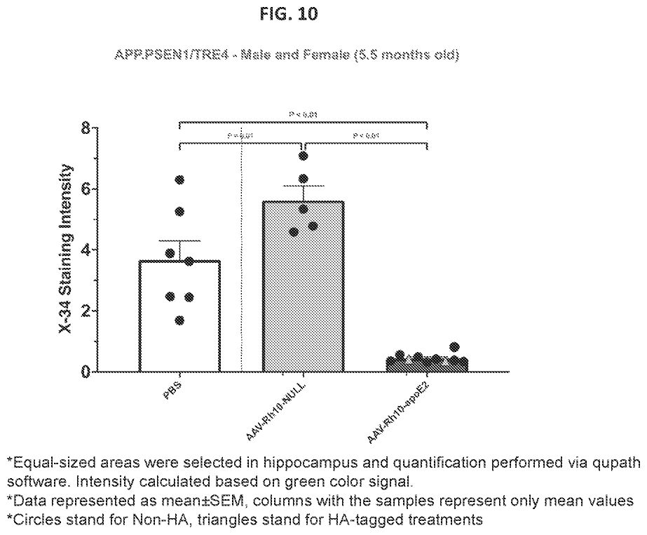

Immunohistochemical staining of the hippocampus for β-amyloid demonstrated a decrease in the number and size of amyloid plaques following AAVrh. 10hAPOE2 administration compared to PBS controls (FIG. 6). Fluoro-Jade staining for neuronal degeneration was decreased from PBS mice to AAVrh.10hAPOE2-treated mice (FIG. 7). Markers of astrogliosis and microgliosis showed a AAVrh.10hAPOE2-dependent decrease in GFAP (FIG. 8) and Ibal (FIG. 9) positive cells. Marker of amyloid aggregate and structure, X-34, depicted a reduction in amyloid levels as measured by a reduction in X-34 stain intensity (FIG. 10).

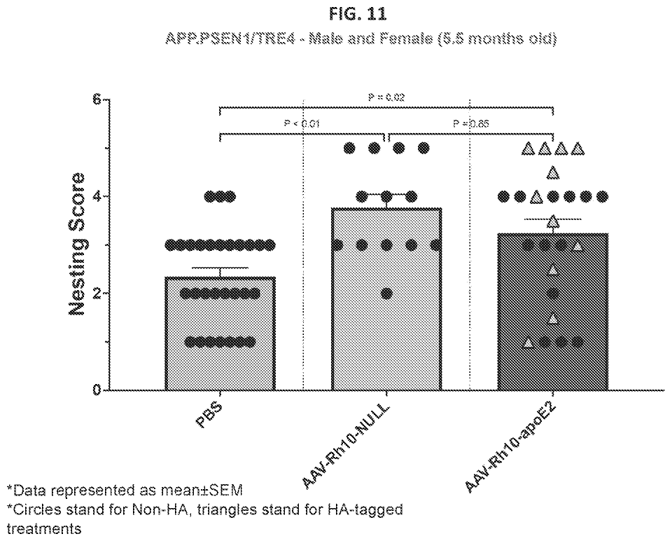

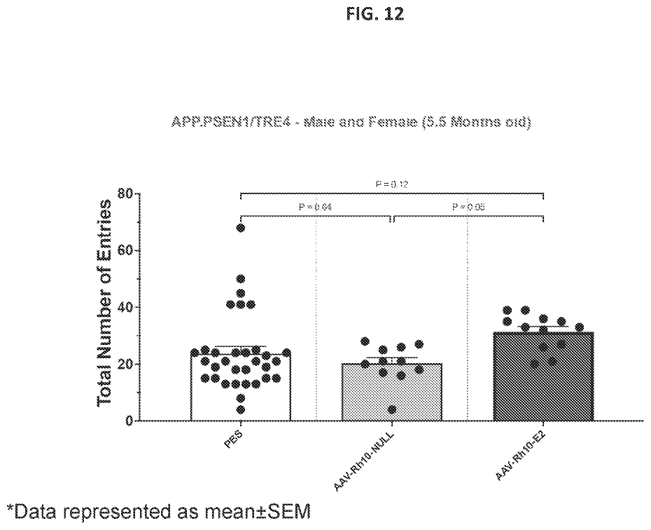

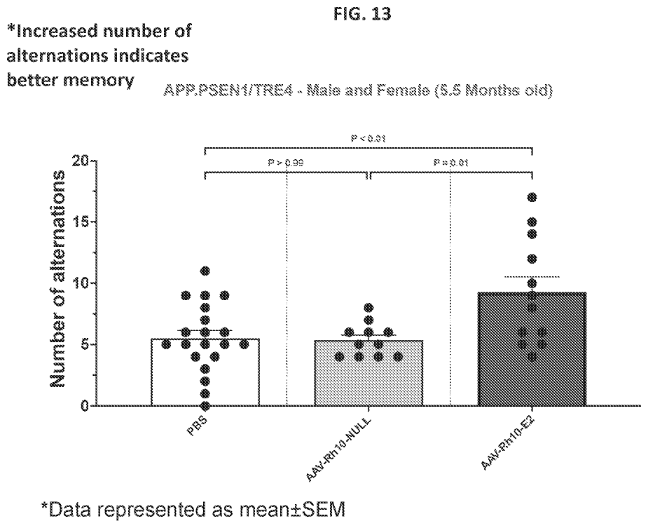

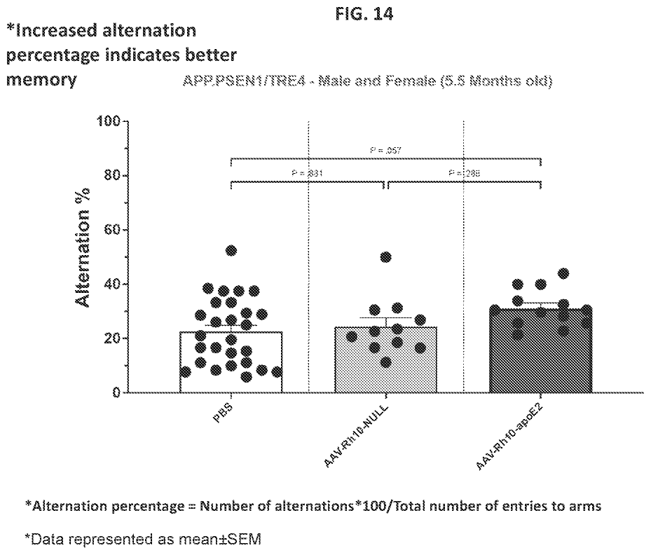

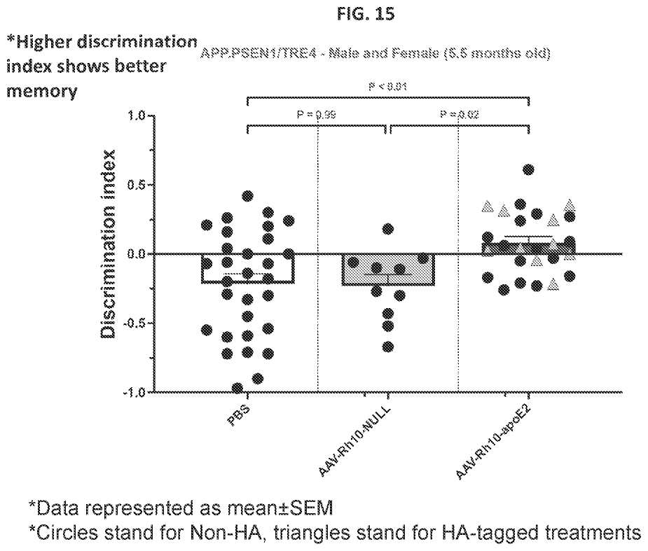

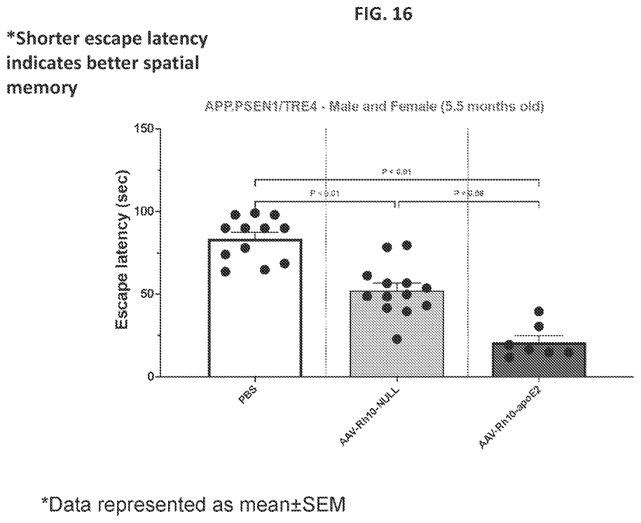

Prior to sacrifice, the mice were assessed by 3 behavioral sensorimotor assays. Compared to PBS, AAVrh.10hAPOE2 treatment: (1) increased the number of alternations and entries in the Y maze (FIG. 12, FIG. 13), FIG. 14; (2) improved the novel object discrimination index (FIG. 15); (3) decreased escape latency in the Barnes maze test (FIGS. 16); and (4) changes in nesting behavior were observed (FIG. 11).

In conclusion, intrahippocampal delivery of AAVrh.10 expressing human APOE2 improves morphologic, biochemical and behavioral disease parameters in the amyloid-driven Alzheimer's mouse.

### Example 3: Manufacture of APOE2 rAAV Viral Vectors

Manufacture of APOE2 rAAV vector pharmaceutical compositions can be performed following the process outlined below.

The APOE2 rAAV vector upstream manufacturing process consists of a series of cell culture expansion steps until transfection of the cells. The production of AAVrh.10hAPOE2 involves the co-transfection of HEK293T cells with two plasmids: AAV vector plasmid (pAAVs-hAPOE2) and the helper plasmid (pPAK-MArh.10). After transfection the cells are incubated until cell harvest. The cell harvest may be stored frozen prior to further processing. The crude viral lysate is recovered from the transfected cells by multiple freeze thaw cycles and residual plasmid DNA and host cell nucleic acids are digested by incubation with Benzonase®. Clarification of the upstream process material to remove cell debris and unbroken cells is conducted by ultracentrifugation. Purification of the process stream is conducted by iodixanol gradients and anion exchange chromatography.

The final processing step is a concentration of the process pool and buffer exchange into the formulation buffer to form the AAVrh.10hAPOE2 drug substance. Sublots may be pooled and then diluted to the target bulk drug substance (BDS) concentration prior to the drug product manufacturing to form a pharmaceutical composition comprising APOE2 rAAV vectors.

### HEK293T WCB Thaw and Expansion

To initiate a batch of AAVrh. 10hAPOE2 a vial of the HEK293T WCB is thawed and propagated by serial culture in DMEM and 10% qualified, gamma irradiated, fetal bovine serum (FBS) without antibiotics or phenol red indicator. The 10% FBS and DMEM media used in the cell expansion phases are preheated prior to use at each stage.

The frozen HEK293T WCB vial is thawed by suspending the cryovial in a beaker containing warmed water for injection (WFI). After visual confirmation of the material thaw, WCB cells are transferred to a 50 ml conical tube containing pre-warmed medium and manually mixed. The HEK293T cells will be grown as adherent culture on the virgin polystyrene surface of Corning 175 cm2 cell culture flasks. The cell suspension from the 50 mL conical tube is transferred into a T75 flask. The T75 flask is incubated in a humidity-controlled incubator at 37° C. and 5% CO2 for up to 48 hours.

After the initial expansion phase the cells are washed with phosphate buffered saline and the cells are detached from the walls of the flask with TrypLE select. Warm 10% FBS DMEM Media is added to the flask to wash the cells from the culture vessel surface and break apart cell clusters. The detached cells suspended in media are transferred to a sterile container and the cell density and viability determined. The cell density is used to calculate the inoculation volume for the next expansion phase. After addition of the target seeding density in the T175 flask, the flask is incubated at 37° C., 90% relative humidity and 5% CO₂, for 2 to 4 days. The cells are expanded until there are sufficient cells for transfection.

Single layer cell stacks and ten (10) layer cell stacks are seeded from the cells recovered from T175 flasks at a target cell density of ≥5E3 cells/cm2. During recovery from the flasks the cell monolayer is washed with PBS, detached from the surfaces with TrypLE select, then resuspended in warm 10% FBS DMEM. Each cell stack is incubated at 37° C., 90% relative humidity and 5% CO2, for up to 4 days prior to transfection.

### PEI-Mediated Transfection

When the cells seeded in cell stacks have grown to a density of approximately 2E5 viable cells/cm2, the AAV vector and helper plasmids will be added to the cells in the presence of PEI (a synthetic transfection reagent not derived from any animal product). The molar ratio of helper to transgene plasmid used in the transfection is approximately 1:2. The transfection mixture is prepared by addition of the calculated volumes of the DNA into a dilution tube with PEI. For each 10-layer cell stack, 1272 μg of Helper plasmid and 636 μg of Transgene plasmid is added into SF-DMEM to dilute the plasmids prior to combining them with 1908 μg of PEI pro in the dilution tube. The solution is mixed and incubated for 15 min at room temperature prior to splitting the prepared transfection mixture to two bottles containing 750 mL each of 2.5% DMEM, for a total complex volume of 36.7 mL per cell stack. Residual media from the expansion phase is removed from the cell stack prior to addition of the transfection mixture.

Transfected cells are cultured in the incubator at 37° C., 90% humidity and 5% CO2 for the production phase.

### Cell Harvest

Three days after transfection, the cells floating in the culture media are harvested from the culture media. The media in the cell stacks are collected and added to centrifuge bottles. The remaining cells are released from the cell stacks using a sterile PBS/EDTA solution, pH 7.5. The cells in the PBS/EDTA solution and floating cells in media are pelleted by centrifugation. The supernatant is discarded and the cell pellet re-suspended in Hypotonic resuspension buffer (20 mM Tris pH 8.0 and 2 mM MgCl2) and pooled. Samples of the cell harvest material (cells and supernatant) are collected for determination of vector genome copy (GC) titer, bioburden and an evaluation of the presence of adventitious agents prior to further processing. The cell harvest will be stored in 50 mL sterile and nonpyrogenic polypropylene conical tubes with screw top lid at ≤−65° C. for up to 3 months prior to further processing.

### Lysis Clarification

The cell harvest is subjected to multiple freeze/thaw cycles to release the target vector from the cells producing crude viral lysate (CVL). For each of the cycles frozen cell harvest is thawed on a heat block, mixed and then placed on dry ice to freeze again. Five freeze/thaw cycles are completed prior to collecting an in-process sample for vector GC titer.

The CVL is treated with a recombinant nuclease (Benzonase) at a target of 200 U/mL in a resuspension buffer containing 2 mM magnesium chloride (MgCl2) to digest any extra viral DNA and the host cell derived nucleic acids. After benzonase addition the CVL is incubated for 60 minutes at 37° C. After benzonase treatment the CVL is clarified by centrifugation to remove cell debris and unbroken cells. After pooling the clarified CVL, an in-process sample is collected and analyzed for vector GC titer.

### Iodixanol Gradient Purification

The clarified CVL is purified using a differential iodixanol density gradient. The separation of AAVrh.10hAPOE2 from cellular components and empty capsids will be achieved at this step using prepared 15%/25%/40%/54% iodixanol solutions in sealed tubes. The tubes are centrifuged with a fixed angle rotor to allow for the gradient separation and the gradient fractions containing full capsids collected. After collection the fractions are diluted with QHP binding buffer (50 mM Tris, 50 mM NaCl, pH 9.0) and stored overnight at 2-8° C. An in-process QC sample of the purified AAVrh. 10hAPOE2 will be taken and analyzed for vector GC titer.

### Anion Exchange Chromatography

Fractions containing the desired AAVrh.10hAPOE2 vector product will be pooled and loaded onto a Hi-Trap QHP anion exchange column. The columns are single use and packed with Q Sepharose High Performance strong quaternary ammonium anion exchange resin. The AAVrh.10hAPOE2 vector is loaded on an equilibrated column and collected in the flow-through. The column is washed with 1 column volume of binding buffer and pooled with the AAVrh.10hAPOE2 flowthrough to recover residual product from the column. Up to two columns are used for purifying the iodixanol fractions collected from a single 10-layer cell stack. An in-process QC sample of the purified AAVrh.10hAPOE2 is collected and analyzed for vector GC titer.

### Ultrafiltration

The AAVrh.10hAPOE2 vector will be concentrated and buffer exchanged using a single use disposable ultrafiltration unit with a 100 kDa MWCO polyetheresulfone (PES) filter to produce a drug substance in its final formulation buffer, phosphate buffered saline. The filter membrane is configured to work with a swinging bucket rotor in a centrifuge. Prior to the initial concentration step the filter membrane is equilibrated with the final formulation buffer. The concentration and buffer exchange operations are controlled by volume gradient markers on the sides of the ultrafiltration unit. Dead stop concentration ratio is limited by filling the collection bottom to the total volume minus the volume of reduction in the sample.

An in-process QC sample of the drug substance will be taken and analyzed for vector GC titer, purity by SDS PAGE, empty capsids, and endotoxin. The drug substance intermediate lots will be stored in 5 mL cryogenic vials, USP VI, at ≤−65° C. for up to 1 year prior to further processing.

### Bulk Drug Substance

Frozen drug substance sublots will be thawed, pooled and adjusted with PBS to

a target concentration that is 10% higher than the target drug product concentration (In some aspects, 1.0E13 gc/mL, but other target drug product concentrations are envisioned and described herein), considering 10% loss during sterile filtration to prepare the drug product. Multiple lots of drug substance may be pooled to form the BDS. The target BDS volume is based upon the quantity of AAVrh.10hAPOE2 vector Drug Product (DP) required to supply an ongoing clinical trial. Pooling and dilution of the drug substance lots is conducted in a sterile single use container immediately prior to sterile filtration and filling of AAVrh.10hAPOE2 vector DP.

Analytical Assessment of AAVrh.10hAPOE2 Vector Pharmaceutical Compositions

Analytical assessment of AAVrh.10hAPOE2 vector pharmaceutical compositions (LX1001) can be performed utilizing the following techniques and methods.

### Transgene Identity

Sanger sequencing of the AAV is performed to confirm the nucleotide sequence of the encapsidated payload. Samples are first treated with proteinase to release the digest AAV capsid proteins, followed by overlapping PCR to fully cover the promoter, enhancer, transgene and poly A regions of the expression cassette. Following PCR, individual bands are purified from an agarose gel and sequenced using Sanger based methods. Bases are assigned using a prevalence-based score and any variations detected are individually reviewed using manual inspection.

### Capsid Purity

Each sample is loaded into an SDS-PAGE gel alongside the reference control based on total viralgenomes calculated by qPCR. The proteins that migrate into the gel are stained with Oriole fluorescent stain. All protein bands in each lane are analyzed for densitometry and the individualimpurity bands are quantified as a percent of the sample. The overall purity is reported for the aggregate VP1, VP2, VP3 quantification as a percent of all bands quantified.

Vector Genome Titer (qPCR)

A Taqman based qPCR assay that targets the CMV portion of the promoter region is used for the quantification of genome copy titer. Samples are initially treated with proteinase K to digest the viral capsid protein coat prior to qPCR analysis. In each assay a positive control AAVrh.10 is included to ensure consistent assay performance and CMV containing reference plasmid (CMV PNY1160) is used to generate a standard curve to quantify the AAV samples tested. Reported DS lot titer is the average of triplicates at two dilution levels. Reported titer for stability DS sample is the average of two independently executed analysis of triplicate samples at two dilution levels.

Vector Genome Titer (ddPCR)

A ddPCR assay was developed to target a region spanning the 5′ end of the transgene encoding region in LX1001 to ensure specificity. Each execution of this method includes a AAVrh.10hAPOE specific reference control to ensure assay performance.

### Infectious Titer

An infectious titer assay will be used to determine the productive uptake and replication of AAVrh.10hAPOE vector in HeLaRC32 cells (rep2 expressing HeLa). This assay measures the ability of the vector particle to infect a reporter cell line and replicate within this line to estimate the infectious capacity of the vector. Serial vector dilutions (10× dilution) are co-infected in replicates of 12 and then infected with adenovirus type 5. Seventy-two hours later, cells are lysedand qPCR performed to detect AAV vector amplification over input. An end-point dilution TCID50 calculation (Spearman-Karber) is performed to determine a replicative titer expressed asTCID50/mL. Since “infectivity” values are dependent on particles coming in contact with cells, they are influenced by assay geometry and are not an absolute measure of the number of “infectious” particles present. However, the ratio of vector genomes to “infectious units” (described as gc: infectious titer ratio) can be used as a measure of product consistency from lot to lot.

### Empty Capsids

To quantify the amount of AAV capsid species present in samples, analytical ultracentrifugationusing sedimentation velocity (SV-AUC) is used. AAV samples are diluted to an A230 nm of 0.8±0.1 and run on a Beckman XLI AUC instrument at 12,000 RPM. The AUC cell is scanned at A230 nm every 90 seconds for 200 total scans, and the scans are then fit in sedfit using a c(s) distribution model over a sedimentation range of 0-200S and resolution of 200. Integration of theresultant peaks and calculation of the area under the c(s) fit profile at the sedimentation coefficient values consistent with empty, partial, full or higher order particle species allows for measurement of relative abundance of the particle populations. To evaluate whether each peak isreproducible or a possible modelling artifact due to the model fitting, a reproducible threshold criterion is implemented. The reproducible threshold is defined as the percentage of absorbance signal for a given species that is <0.5% of the total absorbance and/or <0.002 OD. The result of this analysis is a set of peaks that can be interpreted based on literature precedent and/or additional sample analysis (e.g. by analyzing preparations containing only empty particles). Electron microscopy can also be used to evaluate empty and full AAV capsids.

### Residual Host Cell DNA

A qPCR assay is used to detect residual human DNA from the HEK293 process. DNA is extracted from the test sample and tested using quantitative PCR (qPCR) targeting three separateamplicons in the 18S ribosomal (r)DNA gene. HEK293 derived genomic DNA is used to generate a standard curve to convert the results from a copy number to ng/ml for each amplicon.

### Residual Host Cell Protein

An ELISA is performed to measure levels of contaminating host HEK293 cell proteins. The Cygnus Technologies HEK 293 Host Cell Proteins ELISA kit is used for the analysis. Samples and pre-diluted HEK 293 HCP standards are added to microtiter wells precoated with an affinity purified anti-HEK 293 HCP capture antibody, along with a peroxidase conjugated polyclonal anti-HEK 293 HCP detection antibody. Following incubation, the wells are washed to remove unbound reactants, and TMB, a peroxidase substrate, is added. After development, the reaction is stopped using a sulfuric acid solution. The absorbance of the resulting colored product is measured using a microplate reader, and the amount of HEK 293 HCP in each sample is calculated from the standard curve.

### Residual SV40 Large T Antigen DNA

SV40 containing DNA sequences are measured using quantitative PCR (qPCR). Quantificationis performed relative to a reference standard plasmid containing a single copy of the SV40 LTAsequence. Test samples are run with and without spiking to report the LOD of the assay and assess assay performance.

### Residual Plasmid DNA

Plasmid DNA that is not encoding between the ITR regions is determined in AAV samples by quantitative PCR (qPCR) targeting the kanamycin resistance gene portion of the plasmid sequence. Samples are initially treated with proteinase K to digest the viral capsid protein coat prior to qPCR analysis. Samples are tested both with DNase (treatment prior to proteinase K) to remove any residual non-encapsidated DNA and without, performed in quadruplicate and are reported relative to a known standard.

### Residual E1a DNA

DNA sequences containing the adenovirus Ela genetic element are determined in AAV samples by quantitative PCR (qPCR) using primers and probes specific to the Adenovirus E1a sequence. A standard curve is prepared using genomic DNA extracted from HEK293T cells (which expressE1a). Samples are initially treated with proteinase K to digest the viral capsid protein coat prior to qPCR analysis. Test samples are run with and without spiking to report the LOD of the assay and assess assay performance.

### Residual Benzonase

Benzonase is used in the production process to degrade nucleic acids to facilitate vector purification and as such represents a process impurity. A commercial ELISA is used to measure the concentration of residual Benzonase (EMD Millipore number 1.01681.0002). Samples are analyzed in triplicate wells. Replication Competent AAV

The cell-based assay for replication competence consists of inoculating monolayers of HEK293 cells, an adenovirus (Ad5) transformed cell line and the test article. Due to the presence and co-infection of adenovirus, replication competent AAV will replicate (if present) and amplify in the cell culture. After several days, the viruses will induce cytopathic effects and the cells will be harvested. Cell lysates are used to re-infect fresh monolayers of HEK293 cells two additional times for a total of 3 rounds of amplification. Total cellular DNA is then isolated from the third passage culture and subjected to quantitative PCR (qPCR) analysis. The target used for the analysis is derived from AAV2 Rep sequences, which are required for AAV replication in the presence of adenovirus. The Limit of Detection (LOD) of the assay is 1 rcAAV/1E9 rAAV and the spike controls ensure the absence of inhibition.

### Bioburden

The method is based Membrane Filtration with modifications for quantitative analysis. The sample is separated into two equal portions, filtered onto 0.2 μm membranes, and incubated on TSA and SAB media at 30-35° C. and 20-25° C. respectively for five days to seven days. At the end of the incubation, total number of colonies are counted and divided by the sample volume to yield CFU/mL of sample.

We claim:

1. A pharmaceutical composition comprising APOE2 rAAV viral vectors; wherein the rAAV viral vector comprises an AAVrh10 capsid protein and an APOE2 rAAV vector; wherein the pharmaceutical composition comprises at least about 1.0×10¹¹ genome copies (gc)/mL to about 1.0×10¹⁴ gc/mL and wherein the pharmaceutical composition comprises less than about 40% empty rAAV capsids.

2. The pharmaceutical composition of claim 1, wherein the pharmaceutical composition comprises less than about 40%, about 35%, about 30%, about 25%, about 20%, about 15%, about 10%, or about 5% empty rAAV capsids.

3. The pharmaceutical composition of claim 1, wherein the pharmaceutical composition comprises at least about 1.5×10¹³ gc/mL.

4. The pharmaceutical composition of any one of the preceding claims, wherein the APOE2 rAAV viral vectors are formulated in about 1 mM potassium phosphate monobasic, 3 mM sodium phosphate dibasic, and about 155 mM sodium chloride (NaCl) at a pH of about 7.4.

5. The pharmaceutical composition of any one of the preceding claims, wherein the rAAV vector comprises in the 5′ to 3′ direction:
a first AAV ITR sequence;
an enhancer sequence;
a promoter sequence;
a chimeric intron;
the nucleic acid sequence encoding an apolipoprotein 2 (APOE2) polypeptide;
a polyA sequence; and
a second ITR sequence. 6 The pharmaceutical composition of any one of the preceding claims, wherein the nucleic acid sequence encoding an APOE2 polypeptide comprises SEQ ID NO: 5.

7. The pharmaceutical composition of any one of the preceding claims, wherein the first ITR sequence comprises the nucleic acid sequence set forth in SEQ ID NO: 1.

8. The pharmaceutical composition of any one of the preceding claims, wherein the second ITR sequence comprises the nucleic acid sequence set forth in SEQ ID NO: 7, SEQ ID NO: 10, or SEQ ID NO: 12.

9. The pharmaceutical composition of any one of the preceding claims, wherein the enhancer sequence comprises the nucleic acid sequence set forth in SEQ ID NO: 2.

10. The pharmaceutical composition of any one of the preceding claims, wherein the promoter sequence comprises the nucleic acid sequence set forth in SEQ ID NO: 3.

11. The pharmaceutical composition of any one of the preceding claims, wherein the polyA sequence comprises the nucleic acid sequence set forth in SEQ ID NO: 6.

12. The pharmaceutical composition of any one of the preceding claims, wherein the rAAV vector comprises the nucleic acid sequence set forth in SEQ ID NO: 8 or SEQ ID NO: 11.

13. The pharmaceutical composition of any one of the preceding claims, wherein the rAAV vector is packaged as an rAAV viral vector comprising an AAV capsid protein.

14. The pharmaceutical composition of any one of the preceding claims, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh10 capsid protein.

15. The pharmaceutical composition of any one of the preceding claims, wherein the AAV capsid protein is an AAVrh10 capsid protein.

16. A pharmaceutical composition comprising APOE2 rAAV viral vectors; wherein the rAAV viral vector comprises an AAVrh10 capsid protein and an APOE2 rAAV vector; wherein the APOE2 rAAV vector comprises the nucleic acid sequence set forth in SEQ ID NO: 8 or SEQ ID NO: 11.

17. A method of treating Alzheimer's disease in a subject in need thereof comprising administering a therapeutically effective amount of a pharmaceutical composition according to claim 1 or 16.

18. The method of claim 17, wherein following administration the subject experiences an at least about 5% increase in APOE2 expression relative to a pre-administration baseline.

19. The method of claim 17, wherein the patient is an APOE4 homozygote.

20. The method of claim 17, wherein the pharmaceutical composition is administered via C1-C2 administration or intracisterna magna (ICM) administration.

21. The method of claim 17, wherein the pharmaceutical composition is administered at a dose of about 5.0×10⁹ gc/mL CSF to about 5.0×10¹² gc/mL CSF.

22. The method of claim 17, wherein the pharmaceutical composition is administered at a dose of about:
i) 1.4×10¹⁰ gc/mL CSF,
ii) 4.4×10¹⁰ gc/mL CSF,
iii) 5.0×10¹⁰ gc/mL CSF,
iv) 1.4×10¹⁰ gc/mL CSF,
v) 1.6×10¹⁰ gc/mL CSF, or
vi) 5.0×10¹⁰ gc/mL CSF.

23. The method of claim 17, wherein the pharmaceutical composition is administered in a total volume of about 5 mL, about 10 mL, about 15 mL, or about 20 mL.

24. The method of claim 17, wherein the subject experiences an at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% increase in APOE2 expression.

25. The method of claim 17, wherein the APOE2 expression occurs in the central nervous system.

26. The method of claim 17, wherein the APOE2 expression is measured in the cerebral spinal fluid (CSF).

27. The method of claim 17, wherein following administration of the pharmaceutical composition the expression levels of at least one of T-tau, and P-tau are reduced in the subject relative to a pre-administration baseline.

28. The method of claim 27, wherein the expression levels of T-tau, and/or P-tau are reduced by at least about 5%, at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

29. The method of claim 17, wherein following administration of the pharmaceutical composition the amyloid beta 42/amyloid beta 40 (Aβ_{42/40}) ratio is increased.

30. The method of claim 29, wherein the Aβ_{42/40} ratio is increased by at least about 5%, at least about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

31. The method of claim 17, wherein prior to treatment with the pharmaceutical composition the subject is administered an immunosuppressant.

32. The method of claim 31, wherein the immunosuppressant is prednisone.

33. The method of claim 31, wherein the prednisone is administered at a dosage of:
40 mg, once daily 1 week prior to AAV viral vector administration;
40 mg once daily for week 1 through week 2 post-AAV viral vector administration;
30 mg once daily for week 3 post-AAV viral vector administration;
20 mg once daily for week 4 post-AAV viral vector administration;
10 mg once daily for week 5 post-AAV viral vector administration;
5 mg once daily for week 6 post-AAV viral vector administration;
2.5 mg once daily for week 7 post-AAV viral vector administration; and
2.5 mg every other day for week 8 post-AAV viral vector administration.

34. A method of producing a cellular lysate comprising rAAV viral vectors, the method comprising:
(i) transfecting a cell culture comprising HEK293T cells in a transfection medium with a first plasmid encoding an APOE2 AAV vector and a second plasmid encoding AAV Rep proteins and AAV Cap proteins, wherein the ratio of second plasmid to first plasmid is 2:1;
(ii) culturing the transfected HEK293T cells in the culture medium under conditions in which the transfected HEK293T cells produce recombinant adeno-associated virus (rAAV) viral vectors encoding APOE2;
(iii) harvesting the transfected HEK293T cells; and
(iv) lysing the transfected HEK293T cells to produce a cellular lysate comprising the rAAV viral vectors.

35. The method of claim 34, wherein the culture medium comprises Dulbecco's Modified Eagle Medium (DMEM) with 10% fetal bovine serum (FBS).

36. The method of claim 34, wherein the transfection medium comprises serum-free DMEM and polyethylenimine (PEI).

37. The method of claim 34, wherein the HEK293T cells are obtained after an expansion culture over about two to about five days.

38. The method of claim 34, wherein the transfection of the first plasmid and the second plasmid occur simultaneously.

39. The method of claim 34, wherein the HEK293T cells are present in the culture vessel at a density of between about 2.0×10⁴ to about 2.0×10⁶ cells/cm².

40. The method of claim 34, wherein the transfected cells are cultured for about 3 days.

41. The method of claim 34, wherein the HEK293T cells are lysed via at least about 4 sequential freeze-thaw cycles to produce the cellular lysate.

42. The method of claim 34, wherein the cellular lysate is further treated with a recombinant nuclease to digest any nonencapsidated DNA.

43. The method of claim 34, wherein following DNA digestion the cellular lysate is clarified via ultracentrigufation.

44. The method of any one of claims 34 to 43, wherein the cellular lysate comprises from about 1.0×10⁹ to about 5.0×10¹⁴ genome copies (gc) per milliliter.

45. A method for producing an APOE2 rAAV pharmaceutical composition, the method comprising:
(i) obtaining a cellular lysate comprising rAAV viral vectors encoding APOE2;
(ii) contacting a density gradient with the cellular lysate comprising rAAV viral vectors encoding APOE2 and subjecting the density gradient to centrifugation;
(iii) contacting an anion exchange column with the cellular lysate comprising rAAV viral vectors encoding APOE2;
(iv) eluting the rAAV viral vectors from the column; and
(v) concentrating the eluted rAAV viral vectors via ultrafiltration into a formulation buffer thereby producing an APOE2 rAAV pharmaceutical composition.

46. The method of claim 45, wherein the density gradient is an iodixanol density gradient.

47. The method of claim 45, wherein the iodixanol gradient comprises a step-wise density gradient comprising:
(i) an about 10% to about 20% iodixanol solution;
(ii) an about 20% to about 30% iodixanol solution;
(iii) an about 40% to about 50% iodixanol solution; and
(iv) an about 50% to about 60% iodixanol solution.

48. The method of claim 45, wherein the anion exchange column is a Q sepharose high performance strong quaternary ammonium anion exchange resin column.

49. The method of claim 45, wherein the formulation buffer comprises phosphate buffered saline (PBS).

50. The method of claim 45, wherein the pharmaceutical composition, following ultracentrifugation, comprises about 1.0×10¹⁰ to about 5.0×10¹³ viral genomes per milliliter.

51. An APOE2 rAAV pharmaceutical composition produced by the method of claim 45.

52. A cellular lysate comprising rAAV viral vectors produced by the method of claim 34.

53. An rAAV vector comprising the nucleic acid sequence set forth in SEQ ID NO: 8 or SEQ ID NO: 11.

54. An rAAV vector comprising in the 5′ to 3′ direction:
a first AAV ITR sequence;
an enhancer sequence;
a promoter sequence;
a chimeric intron;
the nucleic acid sequence encoding an apolipoprotein 2 (APOE2) polypeptide;
a polyA sequence; and
a second ITR sequence.


SEQUENCE LISTING (18 sequences)

SEQ ID NO: 1 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 3 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - DNA, 1017 bp, synthetic construct
  source 1..1017: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 5 - DNA, 951 bp, synthetic construct
  source 1..951: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 6 - DNA, 56 bp, synthetic construct
  source 1..56: mol_type = other DNA
aataaaggaaatttattttcattgcaatagtgtgttggaattttttgtgtctctca

SEQ ID NO: 7 - DNA, 129 bp, synthetic construct
  source 1..129: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcc

SEQ ID NO: 8 - DNA, 3620 bp, synthetic construct
  source 1..3620: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagaactagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctttagccgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaatctgttgaagggcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttatcgataccgtcgacctcgagggggggcccggtacccagcttttgttccctttgcggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 9 - protein, 317 aa, Homo sapiens [sequence omitted]

SEQ ID NO: 10 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - DNA, 3631 bp, synthetic construct
  source 1..3631: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagaactagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctttagccgccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaatctgttgaagggcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttatcgataccgtcgacctcgagggggggcccggtacccagcttttgttccctttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 129 bp, synthetic construct
  source 1..129: mol_type = other DNA
ggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 13 - protein, 18 aa, Homo sapiens
MKVLWAALLVTFLAGCQA

SEQ ID NO: 14 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 15 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 16 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 17 - protein, 317 aa, Homo sapiens [sequence omitted]

SEQ ID NO: 18 - protein, 317 aa, Homo sapiens [sequence omitted]